(19) United States
(12) Patent Application Publication
(10) Pub. No.: US 2025/0206725 A1
(43) Pub. Date: Jun. 26, 2025

(54) MODULATORS OF THR-BETA AND METHODS OF USE THEREOF

(71) Applicant: Aligos Therapeutics, Inc., South San Francisco, CA (US)

(72) Inventors: David Craig McGowan, Brussels (BE); Kusum S. Gupta, San Bruno, CA (US)

(73) Assignee: Aligos Therapeutics, Inc., South San Francisco, CA (US)

(21) Appl. No.: 18/990,693

(22) Filed: Dec. 20, 2024

Related U.S. Application Data
(60) Provisional application No. 63/613,423, filed on Dec. 21, 2023.

(51) Int. Cl. C07D 403/12; A61K 31/53; A61K 45/06; C07B 59/00

(52) U.S. Cl. CPC C07D 403/12; A61K 31/53; A61K 45/06; C07B 59/002

(57) ABSTRACT

Disclosed herein are compounds of Formula I:

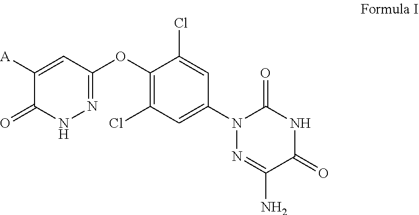

or a stereoisomer or a tautomer thereof, or a pharmaceutically acceptable salt of the compound, the stereoisomer, or the tautomer; wherein A is an isopropyl group, and 1 to 7 hydrogen atoms on the isopropyl group are replaced with deuterium atom(s); pharmaceutical compositions comprising such compounds, and methods of treating disease by administering or contacting a subject with one or more of the above compounds.

### CROSS-REFERENCE TO RELATED APPLICATIONS

This application claims the benefit of priority to U.S. Provisional Patent Application Ser. No. 63/613,423, filed on Dec. 21, 2023, the entire disclosure of which is hereby incorporated by reference herein.

### FIELD OF THE DISCLOSURE

The present disclosure is in the field of pharmaceutical compounds and preparations and method of their use in the treatment of disease. In particular, the present disclosure is in the field of deuterated THR-β modulators and their use.

### BACKGROUND OF THE DISCLOSURE

In parallel with the global increase in obesity, metabolic dysfunction-associated steatotic liver disease (MASLD, formerly known as nonalcoholic fatty liver disease (NAFLD); MASLD and NAFLD are used interchangeably) is becoming the leading cause of chronic liver disease and liver transplantation worldwide [1,2]. MASLD is believed to affect 30% of the adult population and 70-80% of individuals who are obese and diabetic. MASLD is defined as excess liver fat accumulation greater than 5% induced by causes other than alcohol intake. MASLD progresses to liver inflammation (metabolic dysfunction-associated steatohepatitis (MASH), formerly known as nonalcoholic steatohepatitis, NASH; MASH and NASH are used interchangeably) and fibrosis in a variable proportion of individuals, ultimately leading to liver failure and hepatocellular carcinoma (HCC) in susceptible individuals [3].

In the United States alone, MASH is the third most common indication for liver transplantation and is on a trajectory to become the most common [4]. The most important medical need in patients with MASLD and MASH is an effective treatment to halt the progression and possibly reverse fibrosis, which is the main predictor of liver disease evolution [5,6].

Thyroid hormone (TH) is essential for normal development, growth and metabolism of all vertebrates. Its effects are mediated principally through triiodothyronine (T3), which acts as a ligand for the TH receptors (TRs, or THRs) β1, β2 and α1 [7]. In the absence of ligand, TR first binds as a heterodimer or homodimer on TH response elements (TRE) located in the promoter regions of target genes, where it interacts with corepressors. Upon ligand binding, the TR homodimers are dissociated in favor of heterodimer formation with the retinoid-X receptor (RXR), resulting in release of the corepressors and recruitment of coactivators. This new complex attracts a large number of proteins which engage the RNA polymerase II in the transcription of the targeted genes.

Two different genetic loci, denoted THRA and THRB, are responsible for encoding multiple interrelated TR isoforms that have distinct tissue distributions and biological functions. The two major isoforms with the broadest level of tissue expression are TRα1 and TRβ1 [8]. While TRα1 is expressed first during fetal development and is widely expressed in adult tissues, TRβ1 appears later in development and displays highest expression in the adult liver, kidney, and lung [9]. TRα1 is a key regulator of cardiac output, whereas TRβ1 helps in the control of metabolism in the liver. Importantly, the natural thyroid hormone T3 activates both TRα1 and TRβ1 without any significant selectivity.

Design of thyromimetic small molecule agents led to the identification of TR (or THR) agonists with varying levels of TRβ selectivity despite high structural similarity between the ligand-binding domains for TRβ and TRα. TRβ selectivity achieved by some of these compounds resulted in an improved therapeutic index for lipid lowering relative to cardiac effects such as heart rate, cardiac hypertrophy, and contractility [10-12].

TRα and TRβ agonists are also used in indications other than liver-related disorders, as has been known in the art. For example, TRβ selective agonists may be useful in the treatment of X-linked adrenoleukodystrophy [13, 14].

### SUMMARY

Provided herein, in one aspect, are compounds of Formula I:

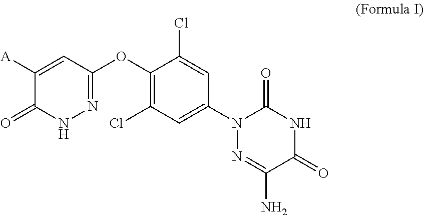

or a stereoisomer or a tautomer thereof, or a pharmaceutically acceptable salt of the compound, the stereoisomer, or the tautomer; wherein A is an isopropyl group, and 1 to 7 hydrogen atoms on the isopropyl group are replaced with deuterium atom(s).

In some embodiments, all 7 hydrogen atoms on the isopropyl group are replaced with deuterium atoms. In some embodiments, 1 to 6 hydrogen atoms on the isopropyl group are replaced with deuterium atoms. In some embodiments, 1 to 4 hydrogen atoms on the isopropyl group are replaced with deuterium atoms.

Provided herein, in another aspect, is a compound selected from the group consisting of:

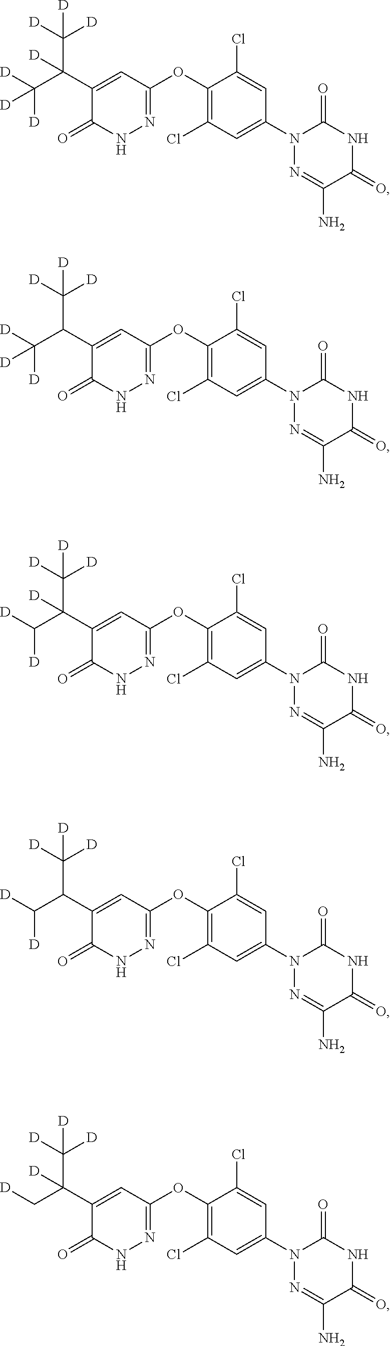 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])([2H])C(c1cc(Oc2c(Cl)cc(-n3nc(N)c(=O)[nH]c3=O)cc2Cl)n[nH]c1=O)C([2H])([2H])[2H] ; [2H]C([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])C(c1cc(Oc2c(Cl)cc(-n3nc(N)c(=O)[nH]c3=O)cc2Cl)n[nH]c1=O)C([2H])([2H])[2H] ; [2H]CC([2H])(c1cc(Oc2c(Cl)cc(-n3nc(N)c(=O)[nH]c3=O)cc2Cl)n[nH]c1=O)C([2H])([2H])[2H]]

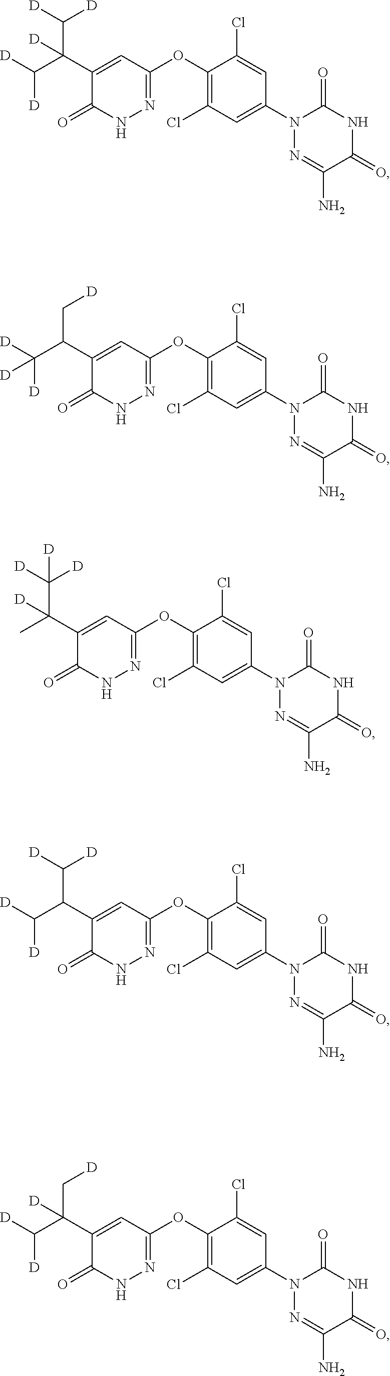 [SMILES: [2H]C([2H])C([2H])(c1cc(Oc2c(Cl)cc(-n3nc(N)c(=O)[nH]c3=O)cc2Cl)n[nH]c1=O)C([2H])[2H] ; [2H]CC(c1cc(Oc2c(Cl)cc(-n3nc(N)c(=O)[nH]c3=O)cc2Cl)n[nH]c1=O)C([2H])([2H])[2H] ; [2H]C([2H])([2H])C([2H])(C)c1cc(Oc2c(Cl)cc(-n3nc(N)c(=O)[nH]c3=O)cc2Cl)n[nH]c1=O ; [2H]C([2H])C(c1cc(Oc2c(Cl)cc(-n3nc(N)c(=O)[nH]c3=O)cc2Cl)n[nH]c1=O)C([2H])[2H] ; [2H]CC([2H])(c1cc(Oc2c(Cl)cc(-n3nc(N)c(=O)[nH]c3=O)cc2Cl)n[nH]c1=O)C([2H])[2H]]

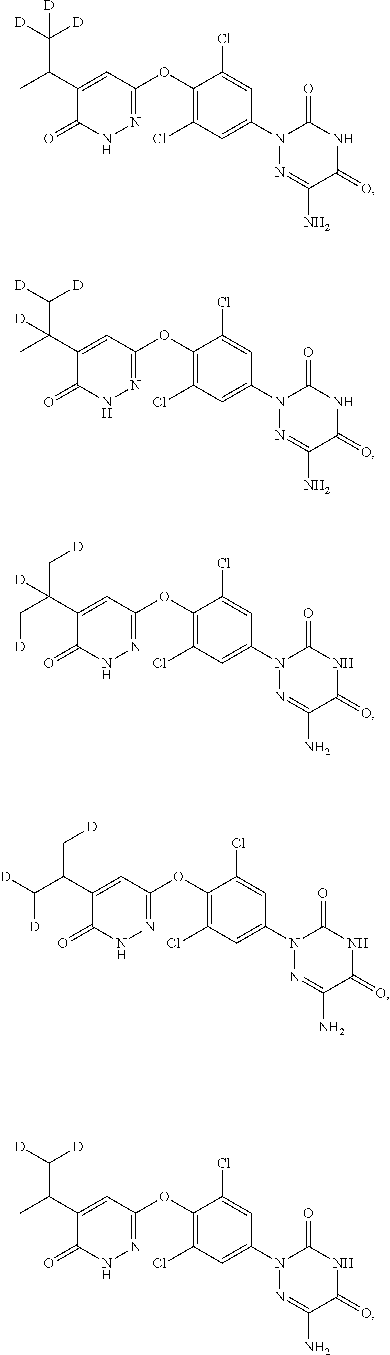 [SMILES: [2H]C([2H])([2H])C(C)c1cc(Oc2c(Cl)cc(-n3nc(N)c(=O)[nH]c3=O)cc2Cl)n[nH]c1=O ; [2H]C([2H])C([2H])(C)c1cc(Oc2c(Cl)cc(-n3nc(N)c(=O)[nH]c3=O)cc2Cl)n[nH]c1=O ; [2H]CC([2H])(C[2H])c1cc(Oc2c(Cl)cc(-n3nc(N)c(=O)[nH]c3=O)cc2Cl)n[nH]c1=O ; [2H]CC(c1cc(Oc2c(Cl)cc(-n3nc(N)c(=O)[nH]c3=O)cc2Cl)n[nH]c1=O)C([2H])[2H] ; [2H]C([2H])C(C)c1cc(Oc2c(Cl)cc(-n3nc(N)c(=O)[nH]c3=O)cc2Cl)n[nH]c1=O]

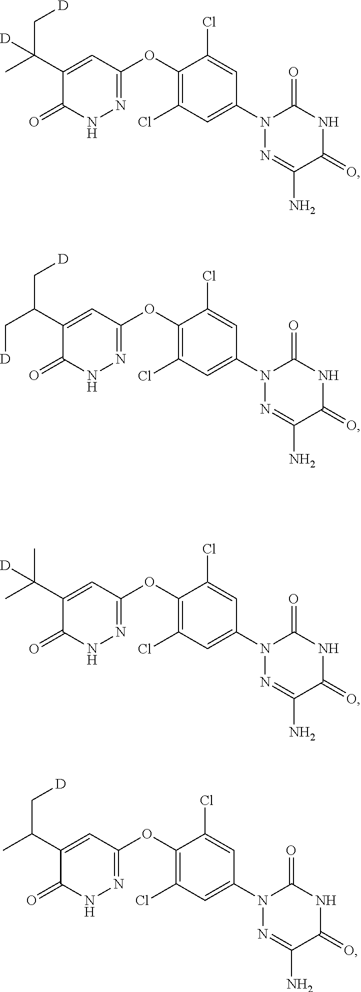 [SMILES: [2H]CC([2H])(C)c1cc(Oc2c(Cl)cc(-n3nc(N)c(=O)[nH]c3=O)cc2Cl)n[nH]c1=O ; [2H]CC(C[2H])c1cc(Oc2c(Cl)cc(-n3nc(N)c(=O)[nH]c3=O)cc2Cl)n[nH]c1=O ; [2H]C(C)(C)c1cc(Oc2c(Cl)cc(-n3nc(N)c(=O)[nH]c3=O)cc2Cl)n[nH]c1=O ; [2H]CC(C)c1cc(Oc2c(Cl)cc(-n3nc(N)c(=O)[nH]c3=O)cc2Cl)n[nH]c1=O]

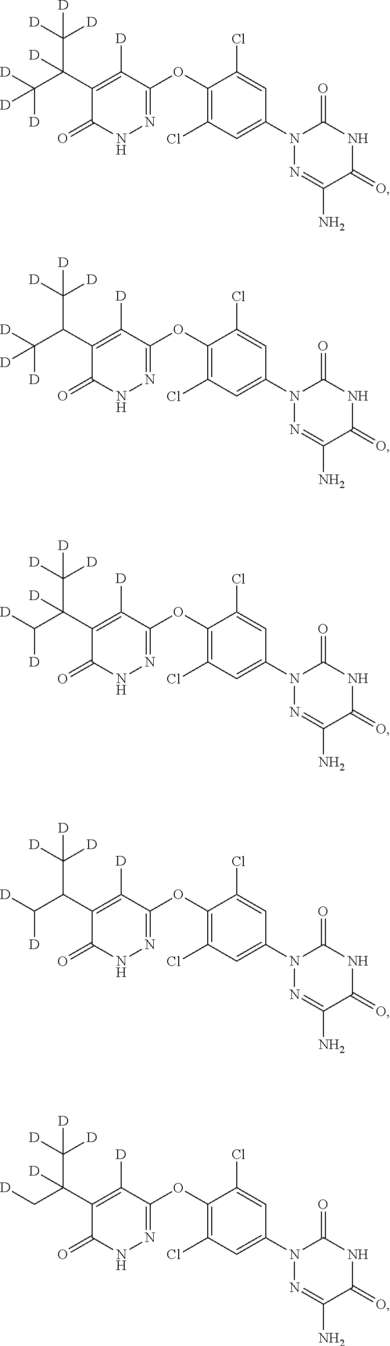 [SMILES: [2H]c1c(Oc2c(Cl)cc(-n3nc(N)c(=O)[nH]c3=O)cc2Cl)n[nH]c(=O)c1C([2H])(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C([2H])(C([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])([2H])[2H] ; [2H]CC([2H])(c1c([2H])c(Oc2c(Cl)cc(-n3nc(N)c(=O)[nH]c3=O)cc2Cl)n[nH]c1=O)C([2H])([2H])[2H]]

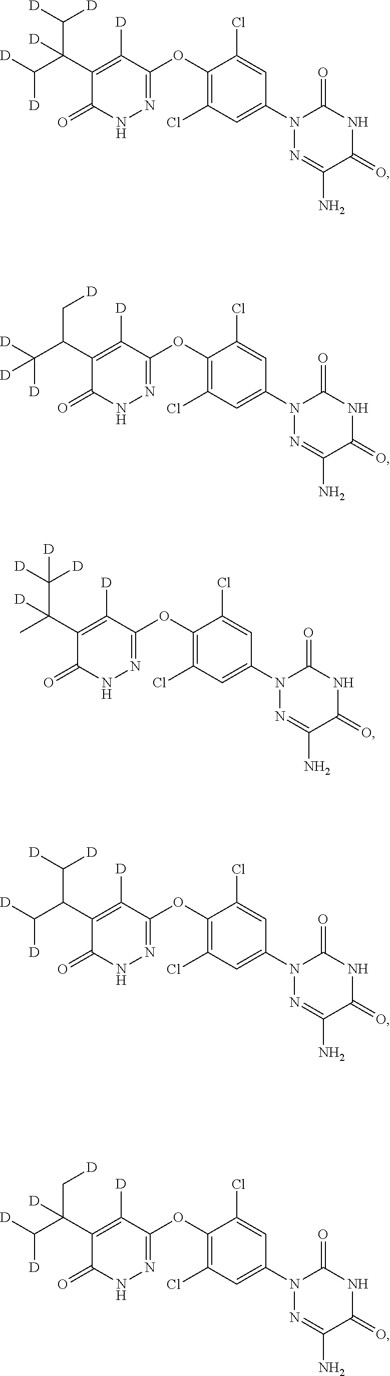 [SMILES: [2H]c1c(Oc2c(Cl)cc(-n3nc(N)c(=O)[nH]c3=O)cc2Cl)n[nH]c(=O)c1C([2H])(C([2H])[2H])C([2H])[2H] ; [2H]CC(c1c([2H])c(Oc2c(Cl)cc(-n3nc(N)c(=O)[nH]c3=O)cc2Cl)n[nH]c1=O)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])[2H] ; [2H]CC([2H])(c1c([2H])c(Oc2c(Cl)cc(-n3nc(N)c(=O)[nH]c3=O)cc2Cl)n[nH]c1=O)C([2H])[2H]]

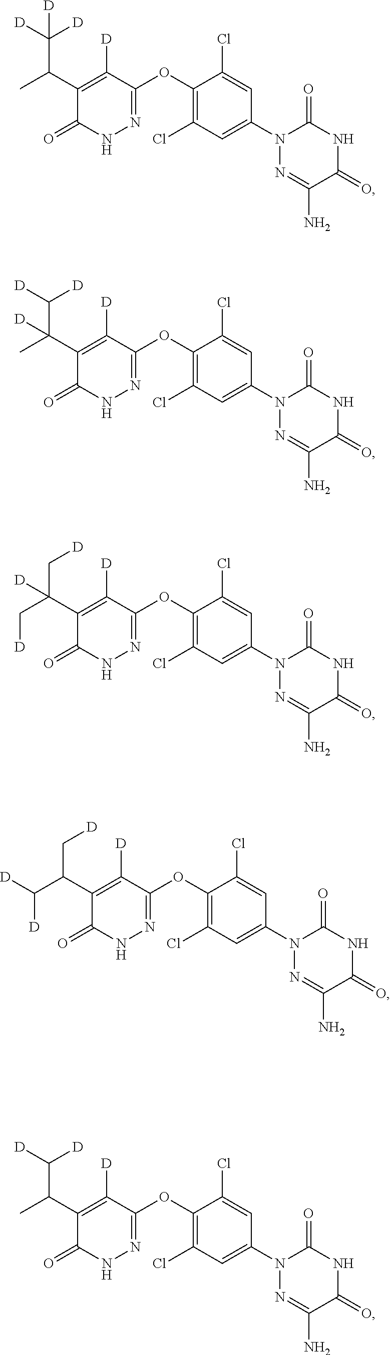 [SMILES: [2H]c1c(Oc2c(Cl)cc(-n3nc(N)c(=O)[nH]c3=O)cc2Cl)n[nH]c(=O)c1C(C)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])[2H] ; [2H]CC([2H])(C[2H])c1c([2H])c(Oc2c(Cl)cc(-n3nc(N)c(=O)[nH]c3=O)cc2Cl)n[nH]c1=O ; [2H]CC(c1c([2H])c(Oc2c(Cl)cc(-n3nc(N)c(=O)[nH]c3=O)cc2Cl)n[nH]c1=O)C([2H])[2H] ; [2H]c1c(Oc2c(Cl)cc(-n3nc(N)c(=O)[nH]c3=O)cc2Cl)n[nH]c(=O)c1C(C)C([2H])[2H]]

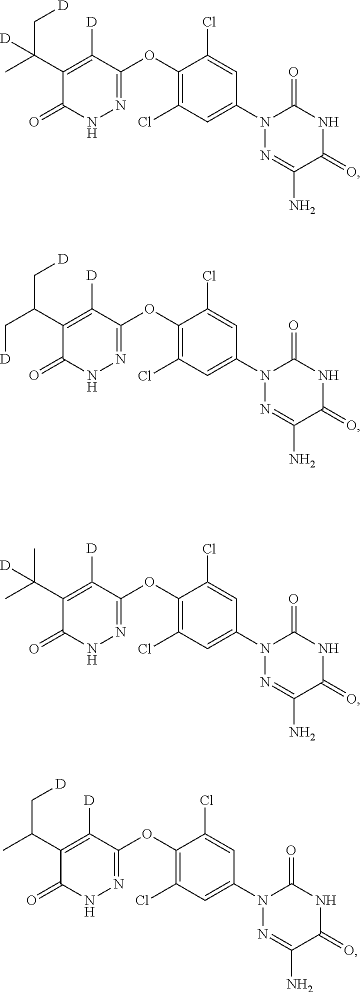 [SMILES: [2H]CC([2H])(C)c1c([2H])c(Oc2c(Cl)cc(-n3nc(N)c(=O)[nH]c3=O)cc2Cl)n[nH]c1=O ; [2H]CC(C[2H])c1c([2H])c(Oc2c(Cl)cc(-n3nc(N)c(=O)[nH]c3=O)cc2Cl)n[nH]c1=O ; [2H]c1c(Oc2c(Cl)cc(-n3nc(N)c(=O)[nH]c3=O)cc2Cl)n[nH]c(=O)c1C([2H])(C)C ; [2H]CC(C)c1c([2H])c(Oc2c(Cl)cc(-n3nc(N)c(=O)[nH]c3=O)cc2Cl)n[nH]c1=O]

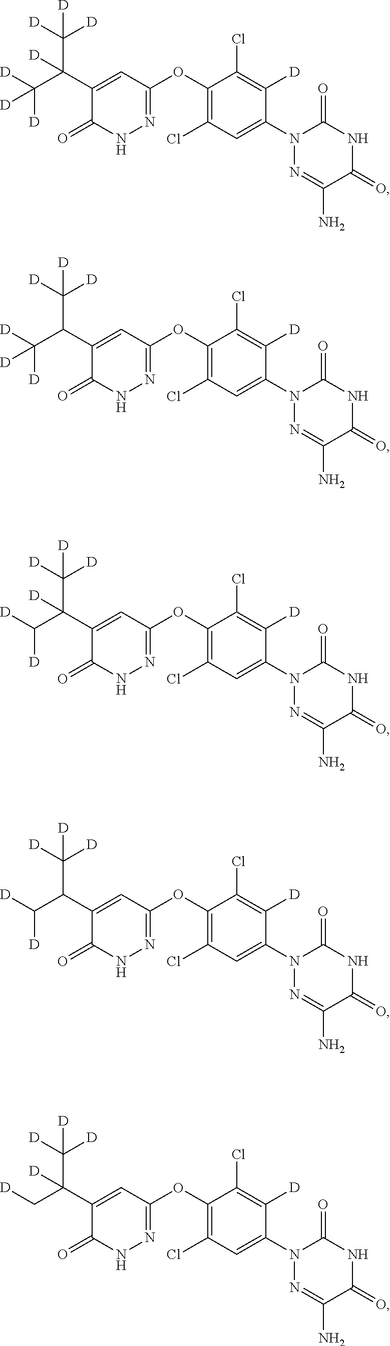 [SMILES: [2H]c1c(-n2nc(N)c(=O)[nH]c2=O)cc(Cl)c(Oc2cc(C([2H])(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C([2H])(C([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])([2H])[2H]]

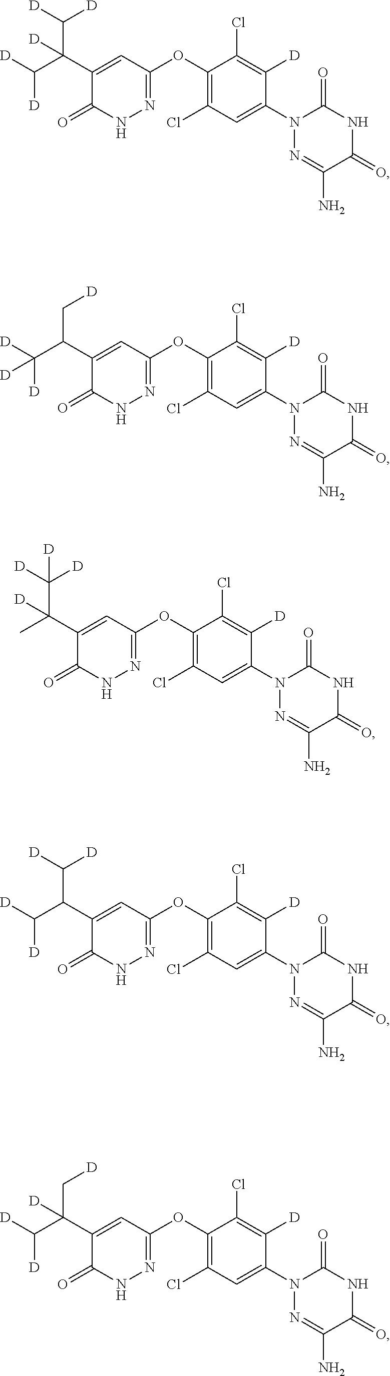 [SMILES: [2H]c1c(-n2nc(N)c(=O)[nH]c2=O)cc(Cl)c(Oc2cc(C([2H])(C([2H])[2H])C([2H])[2H])c(=O)[nH]n2)c1Cl ; [2H]CC(c1cc(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2cc(C([2H])(C)C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])[2H]]

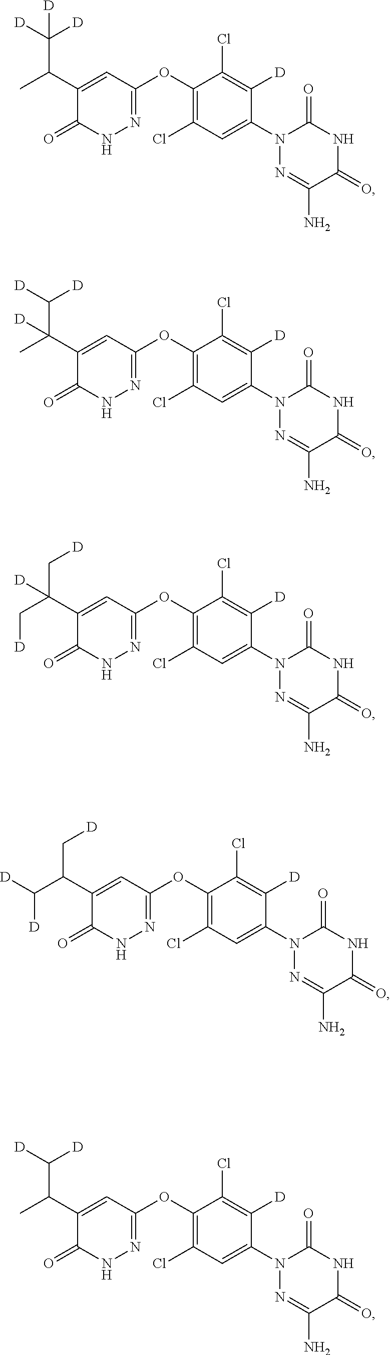 [SMILES: [2H]c1c(-n2nc(N)c(=O)[nH]c2=O)cc(Cl)c(Oc2cc(C(C)C([2H])([2H])[2H])c(=O)[nH]n2)c1Cl ; [2H]c1c(-n2nc(N)c(=O)[nH]c2=O)cc(Cl)c(Oc2cc(C([2H])(C)C([2H])[2H])c(=O)[nH]n2)c1Cl ; [2H]CC([2H])(C[2H])c1cc(Oc2c(Cl)cc(-n3nc(N)c(=O)[nH]c3=O)c([2H])c2Cl)n[nH]c1=O ; [2H]CC(c1cc(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2cc(C(C)C([2H])[2H])c(=O)[nH]n2)c1Cl]

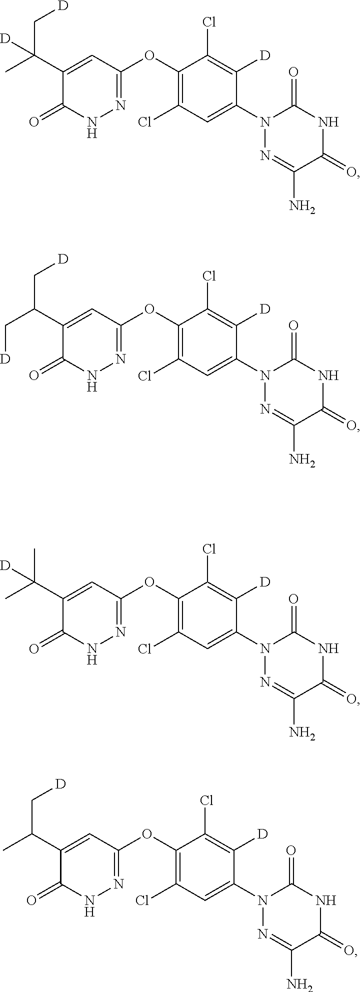 [SMILES: [2H]CC([2H])(C)c1cc(Oc2c(Cl)cc(-n3nc(N)c(=O)[nH]c3=O)c([2H])c2Cl)n[nH]c1=O ; [2H]CC(C[2H])c1cc(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2cc(C([2H])(C)C)c(=O)[nH]n2)c1Cl ; [2H]CC(C)c1cc(Oc2c(Cl)cc(-n3nc(N)c(=O)[nH]c3=O)c([2H])c2Cl)n[nH]c1=O]

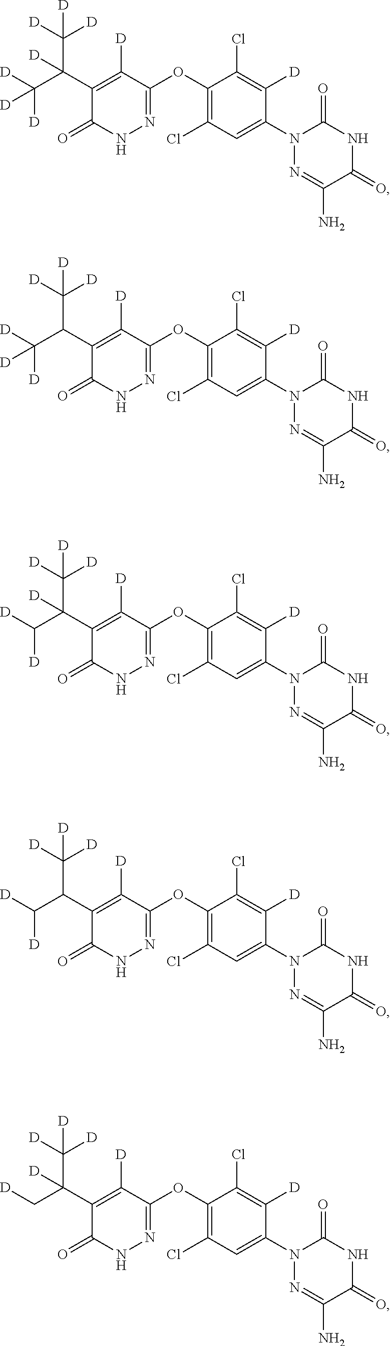

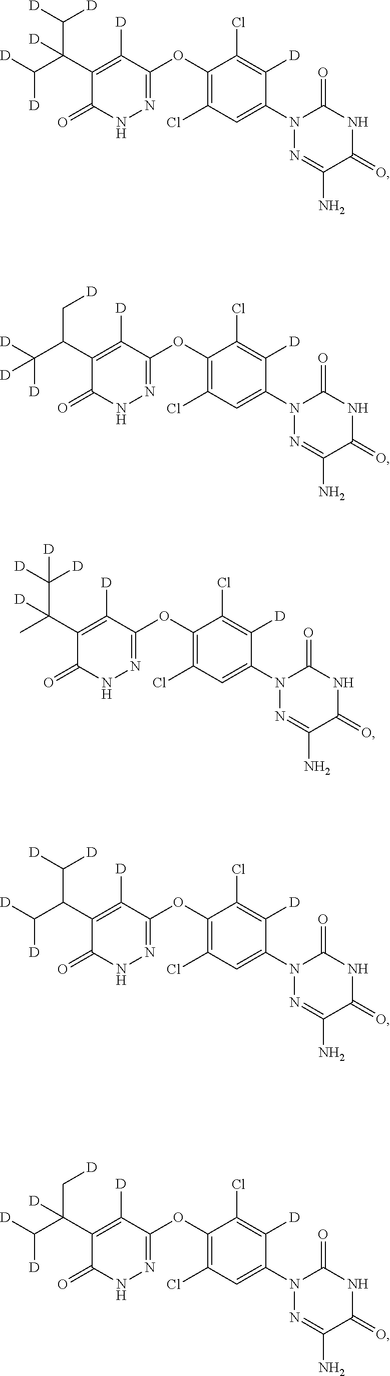 [SMILES: [2H]c1c(-n2nc(N)c(=O)[nH]c2=O)cc(Cl)c(Oc2n[nH]c(=O)c(C([2H])(C([2H])[2H])C([2H])[2H])c2[2H])c1Cl ; [2H]CC(c1c([2H])c(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2n[nH]c(=O)c(C([2H])(C)C([2H])([2H])[2H])c2[2H])c1Cl ; [2H]c1c(-n2nc(N)c(=O)[nH]c2=O)cc(Cl)c(Oc2n[nH]c(=O)c(C(C([2H])[2H])C([2H])[2H])c2[2H])c1Cl ; [2H]CC([2H])(c1c([2H])c(Oc2c(Cl)cc(-n3nc(N)c(=O)[nH]c3=O)c([2H])c2Cl)n[nH]c1=O)C([2H])[2H]]

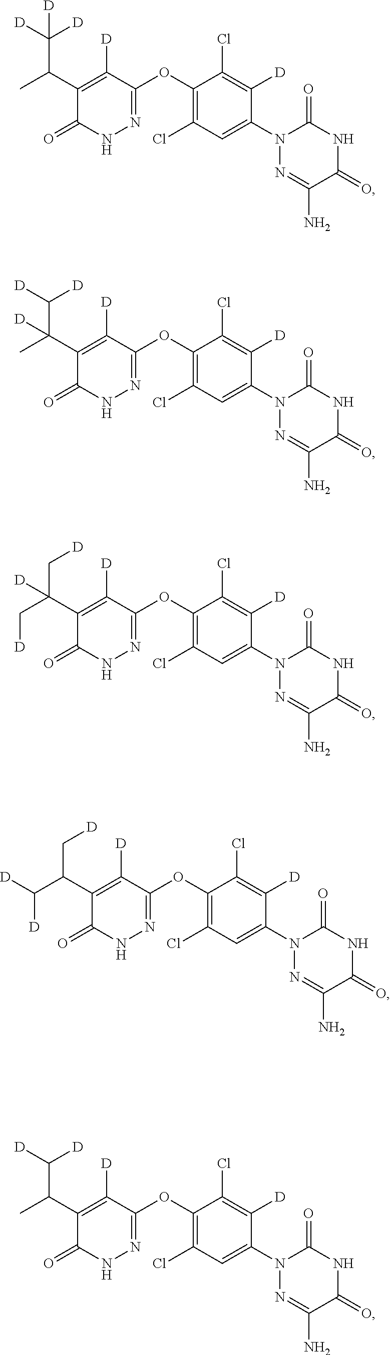 [SMILES: [2H]c1c(-n2nc(N)c(=O)[nH]c2=O)cc(Cl)c(Oc2n[nH]c(=O)c(C(C)C([2H])([2H])[2H])c2[2H])c1Cl ; [2H]c1c(-n2nc(N)c(=O)[nH]c2=O)cc(Cl)c(Oc2n[nH]c(=O)c(C([2H])(C)C([2H])[2H])c2[2H])c1Cl ; [2H]CC([2H])(C[2H])c1c([2H])c(Oc2c(Cl)cc(-n3nc(N)c(=O)[nH]c3=O)c([2H])c2Cl)n[nH]c1=O ; [2H]CC(c1c([2H])c(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2n[nH]c(=O)c(C(C)C([2H])[2H])c2[2H])c1Cl]

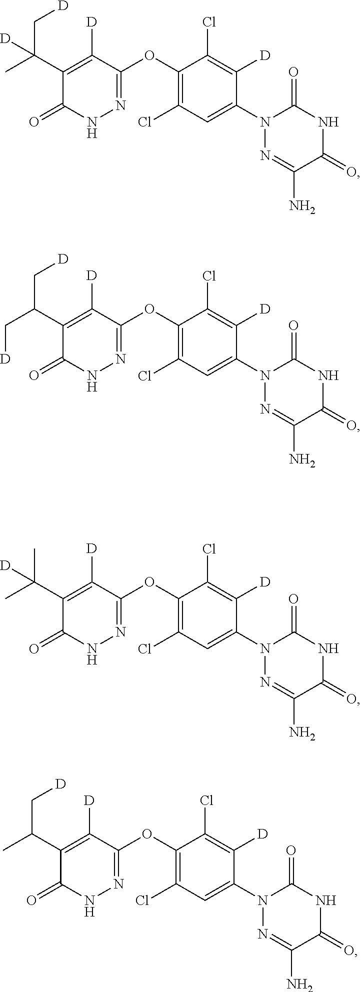 [SMILES: [2H]CC([2H])(C)c1c([2H])c(Oc2c(Cl)cc(-n3nc(N)c(=O)[nH]c3=O)c([2H])c2Cl)n[nH]c1=O ; [2H]CC(C[2H])c1c([2H])c(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2n[nH]c(=O)c(C([2H])(C)C)c2[2H])c1Cl ; [2H]CC(C)c1c([2H])c(Oc2c(Cl)cc(-n3nc(N)c(=O)[nH]c3=O)c([2H])c2Cl)n[nH]c1=O]

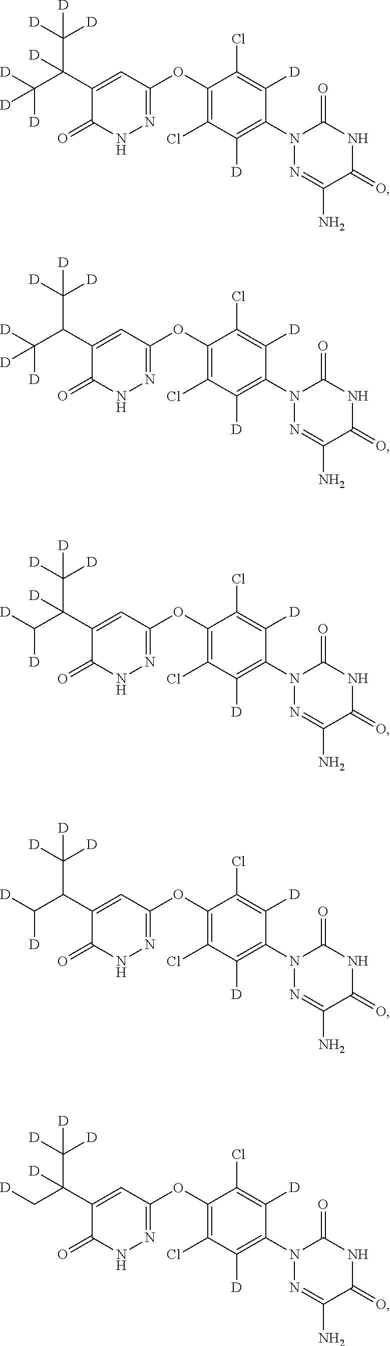

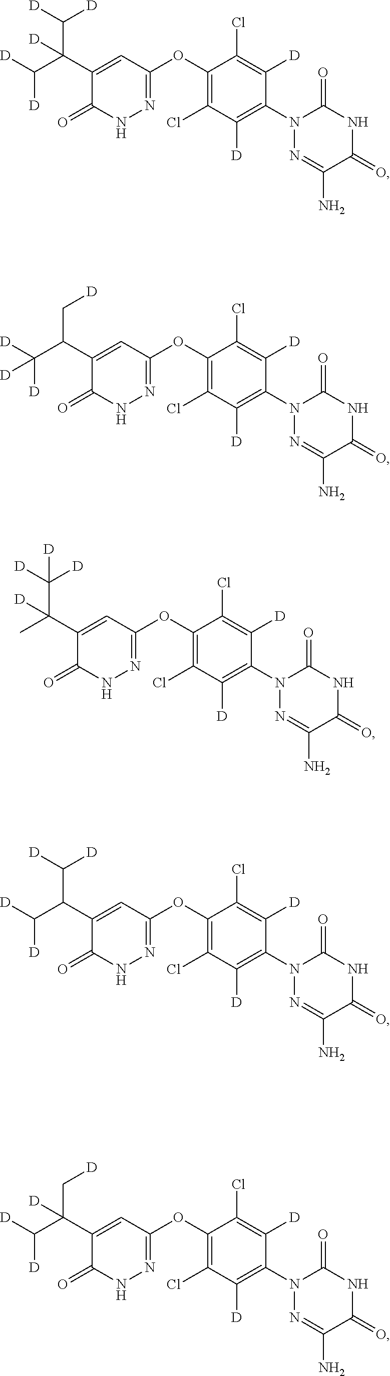 [SMILES: [2H]c1c(Cl)c(Oc2cc(C([2H])(C([2H])[2H])C([2H])[2H])c(=O)[nH]n2)c(Cl)c([2H])c1-n1nc(N)c(=O)[nH]c1=O ; [2H]CC(c1cc(Oc2c(Cl)c([2H])c(-n3nc(N)c(=O)[nH]c3=O)c([2H])c2Cl)n[nH]c1=O)C([2H])([2H])[2H] ; [2H]c1c(Cl)c(Oc2cc(C([2H])(C)C([2H])([2H])[2H])c(=O)[nH]n2)c(Cl)c([2H])c1-n1nc(N)c(=O)[nH]c1=O ; [2H]c1c(Cl)c(Oc2cc(C(C([2H])[2H])C([2H])[2H])c(=O)[nH]n2)c(Cl)c([2H])c1-n1nc(N)c(=O)[nH]c1=O ; [2H]CC([2H])(c1cc(Oc2c(Cl)c([2H])c(-n3nc(N)c(=O)[nH]c3=O)c([2H])c2Cl)n[nH]c1=O)C([2H])[2H]]

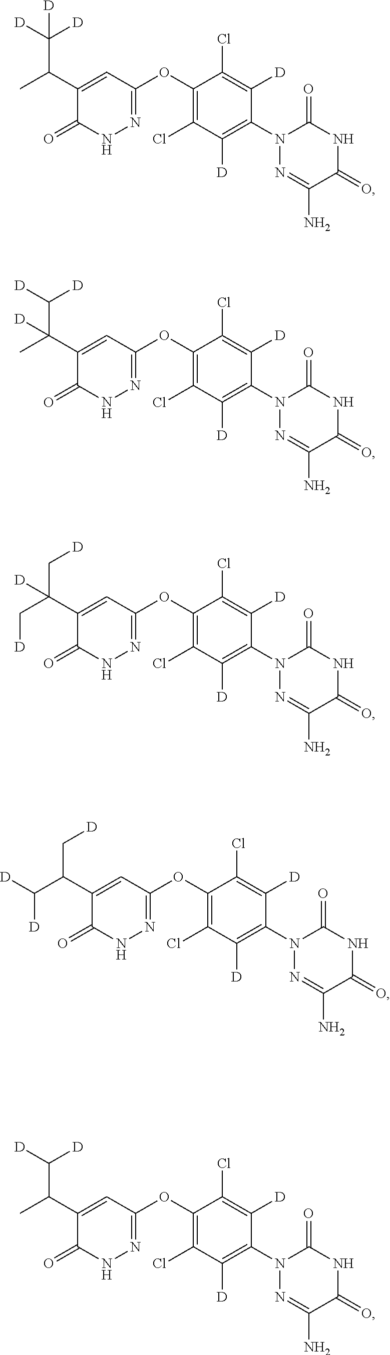 [SMILES: [2H]c1c(Cl)c(Oc2cc(C(C)C([2H])([2H])[2H])c(=O)[nH]n2)c(Cl)c([2H])c1-n1nc(N)c(=O)[nH]c1=O ; [2H]c1c(Cl)c(Oc2cc(C([2H])(C)C([2H])[2H])c(=O)[nH]n2)c(Cl)c([2H])c1-n1nc(N)c(=O)[nH]c1=O ; [2H]CC([2H])(C[2H])c1cc(Oc2c(Cl)c([2H])c(-n3nc(N)c(=O)[nH]c3=O)c([2H])c2Cl)n[nH]c1=O ; [2H]CC(c1cc(Oc2c(Cl)c([2H])c(-n3nc(N)c(=O)[nH]c3=O)c([2H])c2Cl)n[nH]c1=O)C([2H])[2H] ; [2H]c1c(Cl)c(Oc2cc(C(C)C([2H])[2H])c(=O)[nH]n2)c(Cl)c([2H])c1-n1nc(N)c(=O)[nH]c1=O]

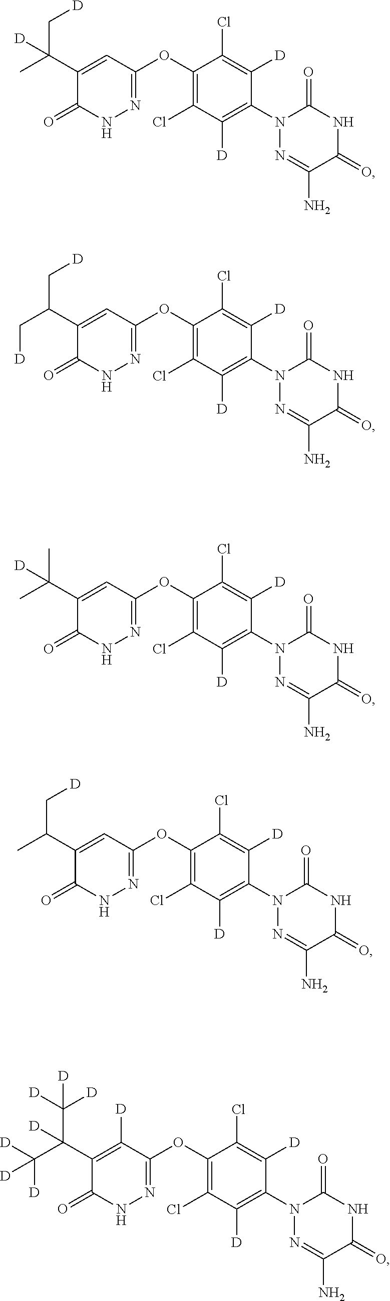 [SMILES: [2H]CC([2H])(C)c1cc(Oc2c(Cl)c([2H])c(-n3nc(N)c(=O)[nH]c3=O)c([2H])c2Cl)n[nH]c1=O ; [2H]CC(C[2H])c1cc(Oc2c(Cl)c([2H])c(-n3nc(N)c(=O)[nH]c3=O)c([2H])c2Cl)n[nH]c1=O ; [2H]c1c(Cl)c(Oc2cc(C([2H])(C)C)c(=O)[nH]n2)c(Cl)c([2H])c1-n1nc(N)c(=O)[nH]c1=O ; [2H]CC(C)c1cc(Oc2c(Cl)c([2H])c(-n3nc(N)c(=O)[nH]c3=O)c([2H])c2Cl)n[nH]c1=O ; [2H]c1c(Cl)c(Oc2n[nH]c(=O)c(C([2H])(C([2H])([2H])[2H])C([2H])([2H])[2H])c2[2H])c(Cl)c([2H])c1-n1nc(N)c(=O)[nH]c1=O]

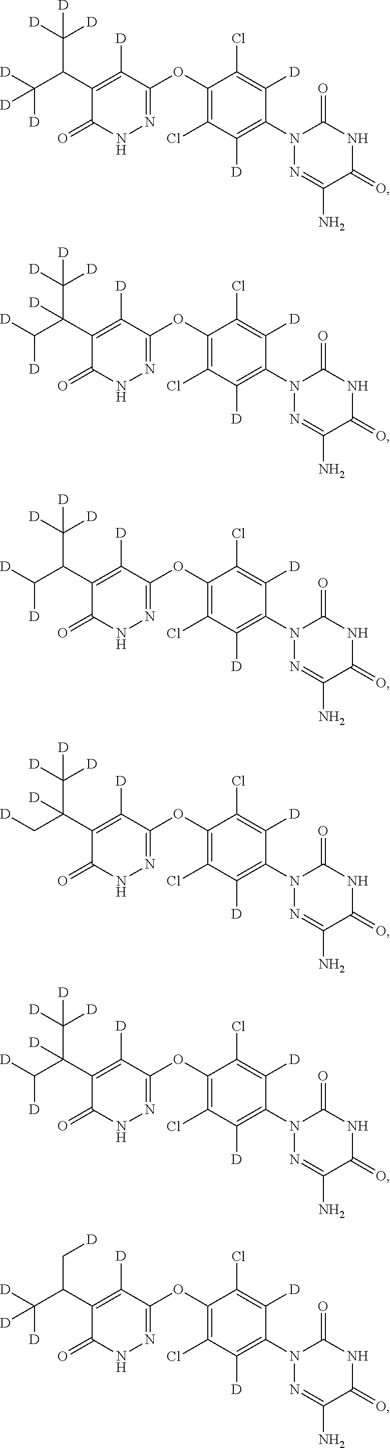

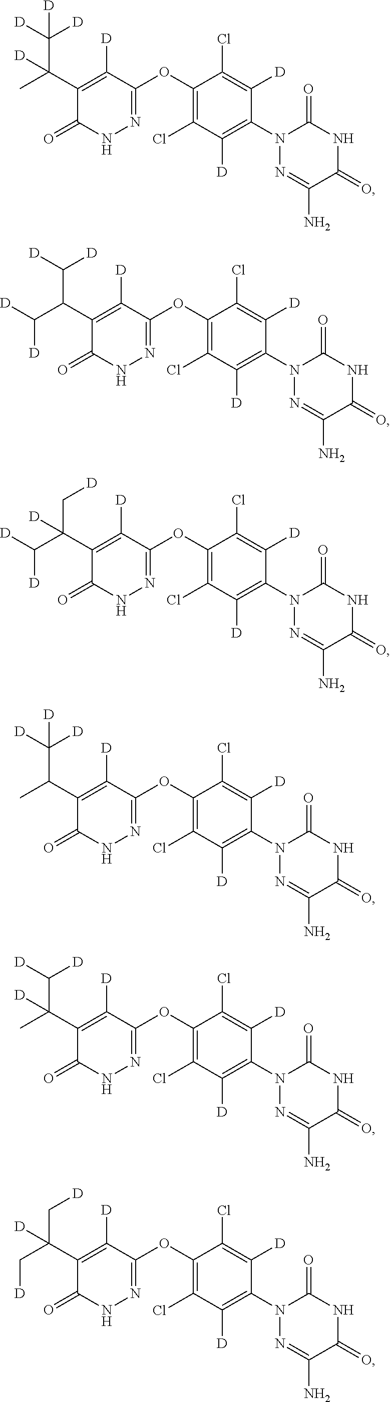

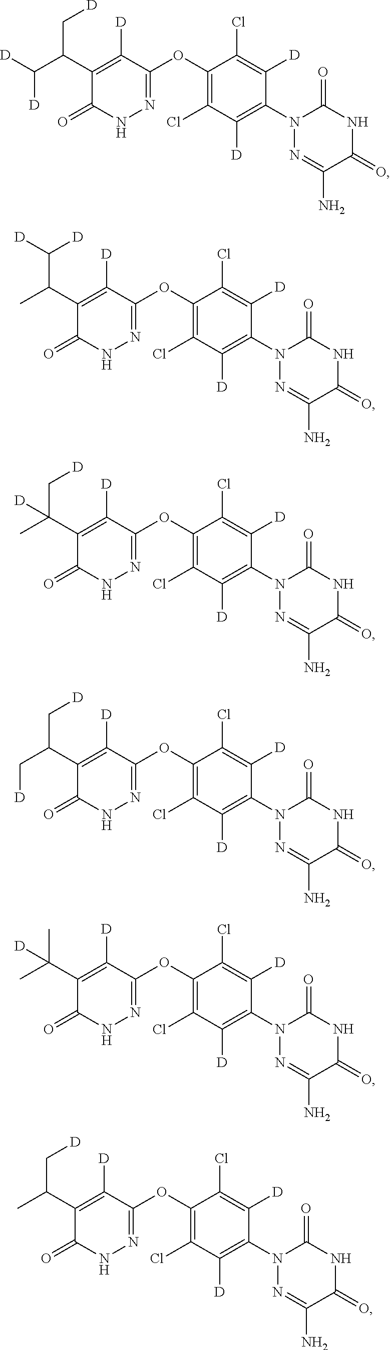

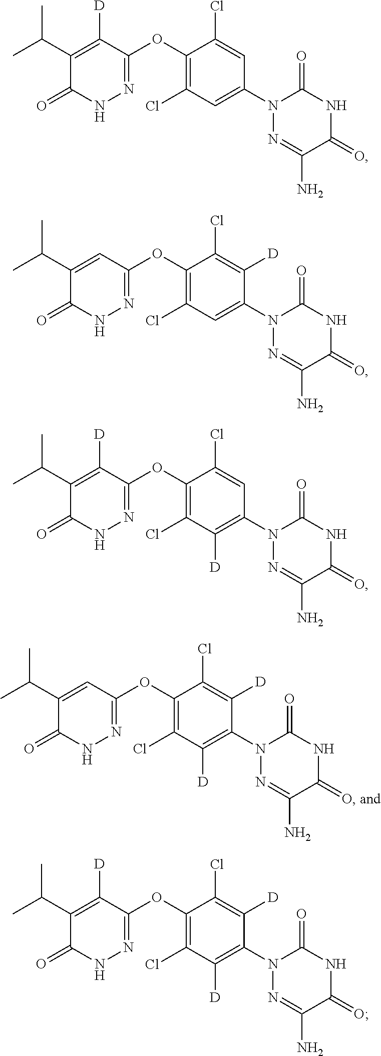

a stereoisomer or a tautomer thereof, or a pharmaceutically acceptable salt of the compound, the stereoisomer, or the tautomer. In some embodiments, the compound is

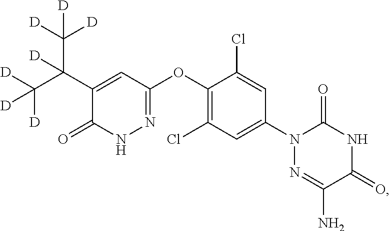 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H]]

or a stereoisomer or a tautomer thereof, or a pharmaceutically acceptable salt of the compound, the stereoisomer, or the tautomer.

Provided herein, in another aspect, is a pharmaceutical composition comprising a compound disclosed herein and at least one pharmaceutically acceptable excipient.

Provided herein, in another aspect, is a method of treating a disorder or disease in a subject in need thereof, the method comprising administering to the subject a therapeutically effective amount of a compound disclosed herein or a therapeutically effective amount of a pharmaceutical composition disclosed herein, wherein the disorder or disease is selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a use of a compound disclosed herein for the manufacture of a medicament for the treatment of a disorder or disease selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a compound disclosed herein for use in treating a disorder or disease selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a composition disclosed herein for use in treating a disorder or disease selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a method of treating a thyroid hormone receptor related disorder in a patient, the method comprising the steps of identifying a patient in need of treatment for the thyroid hormone receptor related disorder, and administering to the patient, or contacting the patient with, a compound disclosed herein or a therapeutically effective amount of a pharmaceutical composition disclosed herein. In some embodiments, the thyroid hormone receptor related disorder is selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer. In some embodiments, the compound disclosed herein or the pharmaceutical composition disclosed herein is administered in combination with a KHK inhibitor, an FXR agonist, a SSAO inhibitor, a FASN inhibitor, or a SCD1 modulator. In some embodiments, the KHK inhibitor is PF-06835919; the FXR agonist is TERN-101 (LY2562175), tropifexor, obeticholic acid (OCA), or ASC42; the SSAO inhibitor is TERN-201; the FASN inhibitor is ASC40; and the SCD1 modulator is aramchol.

Provided herein, in another aspect, is a method of selectively modulating the activity of a thyroid hormone receptor beta (THR-β) comprising contacting a compound disclosed herein with the thyroid hormone receptor. In some embodiments, the contacting is in vitro or ex vivo. In some embodiments, the contacting is in vivo.

Provided herein, in another aspect, is a compound disclosed herein for use in selectively modulating the activity of a thyroid hormone receptor beta (THR-β).

Provided herein, in another aspect, is a composition disclosed herein for use in selectively modulating the activity of a thyroid hormone receptor beta (THR-β).

### DETAILED DESCRIPTION

### Definitions

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, the abbreviations “TR” and “THR” refer to thyroid hormone receptors.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methane-sulfonates, ethanesulfonates, p-toluenesulfonates and salicylates.

Where the compounds disclosed herein have at least one chiral center, they may exist as a racemate or as individual enantiomers. It should be noted that all such isomers and mixtures thereof are included in the scope of the present disclosure. Thus, the illustration of a chiral center without a designation of R or S signifies that the scope of the disclosure includes the R isomer, the S isomer, the racemic mixture of the isomers, or mixtures where one isomer is present in greater abundance than the other.

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid, followed by fractional crystallization and regeneration of the free base. The compounds may also be resolved by formation of diastereomeric esters or amides followed by chromatographic separation and removal of the chiral auxiliary.

It is understood that, in any compound of the presently disclosed compounds having one or more chiral centers, if an absolute stereochemistry is not expressly indicated, then each center may independently be R or S or a mixture thereof. In addition, it is understood that, in any compound of the presently disclosed compounds having one or more double bond(s) generating geometrical isomers that can be defined as E or Z each double bond may independently be E or Z, or a mixture thereof.

The term “pharmaceutical composition” refers to a mixture of one or more compounds disclosed herein with one or more other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example, dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

In certain embodiments, the same substance can act as a carrier, diluent, or excipient, or have any of the two roles, or have all three roles. Thus, a single additive to the pharmaceutical composition can have multiple functions.

The term “pharmaceutically acceptable” defines a carrier or diluent that does not abrogate the biological activity and properties of the compound.

### Compounds

In one aspect, provided herein are compounds of Formula I:

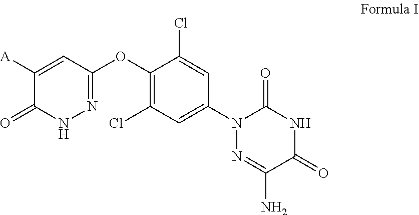

or a stereoisomer or a tautomer thereof, or a pharmaceutically acceptable salt of the compound, the stereoisomer, or the tautomer; wherein A is an isopropyl group, and 1 to 7 hydrogen atoms on the isopropyl group are replaced with deuterium atom(s).

In some embodiments, all 7 hydrogen atoms on the isopropyl group are replaced with deuterium atoms. In some embodiments, 1 to 6 hydrogen atoms on the isopropyl group are replaced with deuterium atoms. This includes 1, 2, 3, 4, 5, or 6 hydrogen atoms on the isopropyl group. In some embodiments, 1 to 5 hydrogen atoms on the isopropyl group are replaced with deuterium atoms. In some embodiments, 1 to 4 hydrogen atoms on the isopropyl group are replaced with deuterium atoms. In some embodiments, 1 to 3 hydrogen atoms on the isopropyl group are replaced with deuterium atoms. In some embodiments, 1 or 2 hydrogen atoms on the isopropyl group are replaced with deuterium atoms.

In another aspect, disclosed herein is a compound selected from the group consisting of:

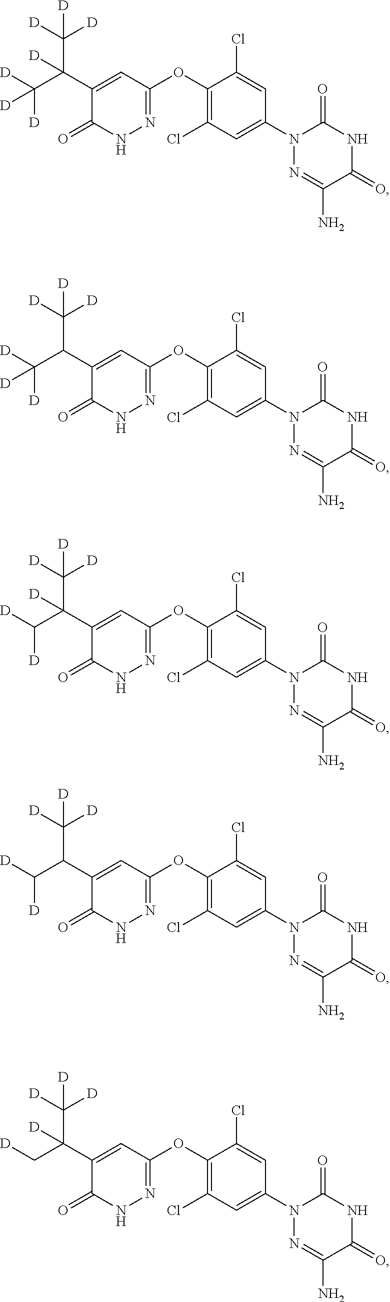 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])([2H])C(c1cc(Oc2c(Cl)cc(-n3nc(N)c(=O)[nH]c3=O)cc2Cl)n[nH]c1=O)C([2H])([2H])[2H] ; [2H]C([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])C(c1cc(Oc2c(Cl)cc(-n3nc(N)c(=O)[nH]c3=O)cc2Cl)n[nH]c1=O)C([2H])([2H])[2H] ; [2H]CC([2H])(c1cc(Oc2c(Cl)cc(-n3nc(N)c(=O)[nH]c3=O)cc2Cl)n[nH]c1=O)C([2H])([2H])[2H]]

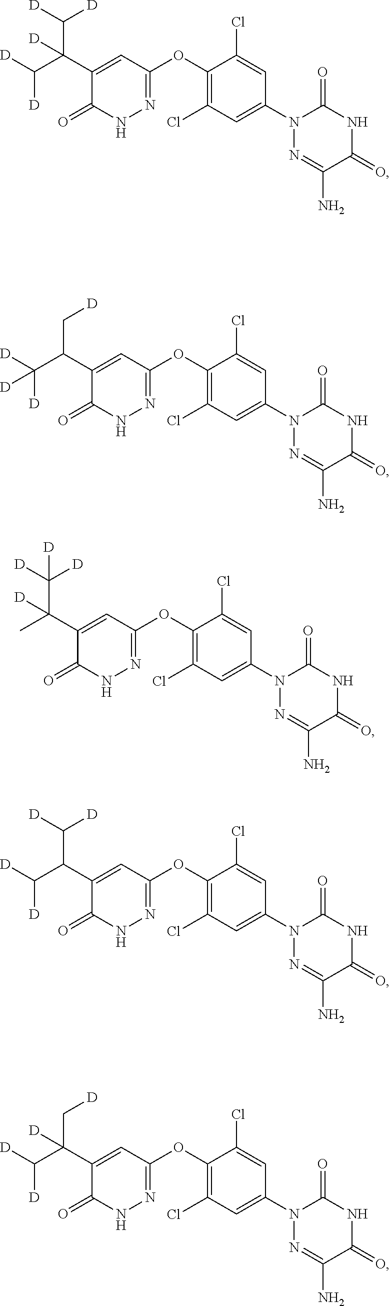 [SMILES: [2H]C([2H])C([2H])(c1cc(Oc2c(Cl)cc(-n3nc(N)c(=O)[nH]c3=O)cc2Cl)n[nH]c1=O)C([2H])[2H] ; [2H]CC(c1cc(Oc2c(Cl)cc(-n3nc(N)c(=O)[nH]c3=O)cc2Cl)n[nH]c1=O)C([2H])([2H])[2H] ; [2H]C([2H])([2H])C([2H])(C)c1cc(Oc2c(Cl)cc(-n3nc(N)c(=O)[nH]c3=O)cc2Cl)n[nH]c1=O ; [2H]C([2H])C(c1cc(Oc2c(Cl)cc(-n3nc(N)c(=O)[nH]c3=O)cc2Cl)n[nH]c1=O)C([2H])[2H] ; [2H]CC([2H])(c1cc(Oc2c(Cl)cc(-n3nc(N)c(=O)[nH]c3=O)cc2Cl)n[nH]c1=O)C([2H])[2H]]

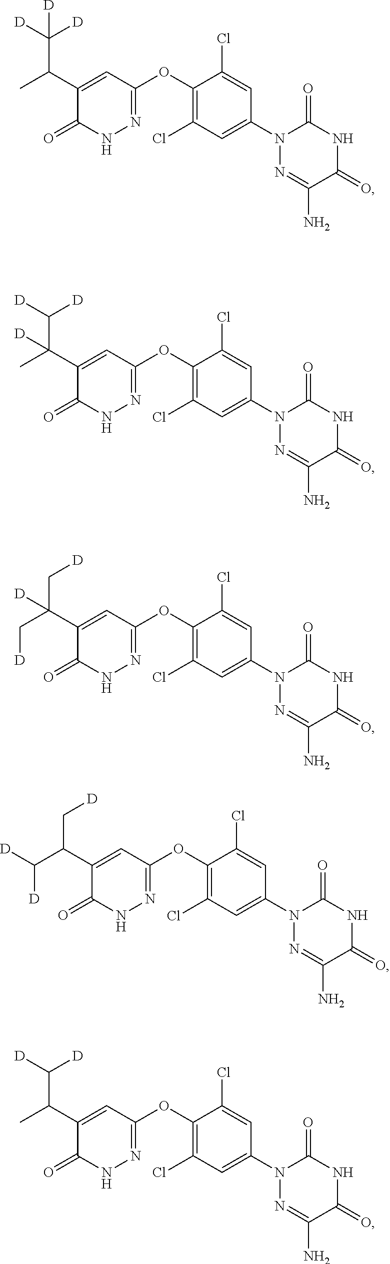 [SMILES: [2H]C([2H])([2H])C(C)c1cc(Oc2c(Cl)cc(-n3nc(N)c(=O)[nH]c3=O)cc2Cl)n[nH]c1=O ; [2H]C([2H])C([2H])(C)c1cc(Oc2c(Cl)cc(-n3nc(N)c(=O)[nH]c3=O)cc2Cl)n[nH]c1=O ; [2H]CC([2H])(C[2H])c1cc(Oc2c(Cl)cc(-n3nc(N)c(=O)[nH]c3=O)cc2Cl)n[nH]c1=O ; [2H]CC(c1cc(Oc2c(Cl)cc(-n3nc(N)c(=O)[nH]c3=O)cc2Cl)n[nH]c1=O)C([2H])[2H] ; [2H]C([2H])C(C)c1cc(Oc2c(Cl)cc(-n3nc(N)c(=O)[nH]c3=O)cc2Cl)n[nH]c1=O]

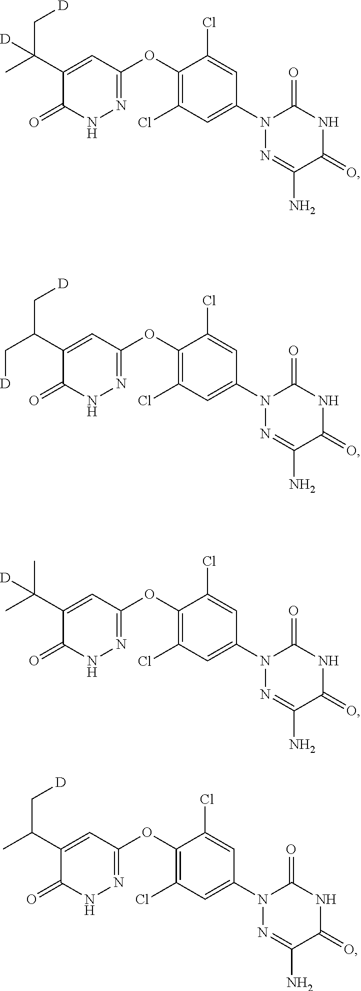 [SMILES: [2H]CC([2H])(C)c1cc(Oc2c(Cl)cc(-n3nc(N)c(=O)[nH]c3=O)cc2Cl)n[nH]c1=O ; [2H]CC(C[2H])c1cc(Oc2c(Cl)cc(-n3nc(N)c(=O)[nH]c3=O)cc2Cl)n[nH]c1=O ; [2H]C(C)(C)c1cc(Oc2c(Cl)cc(-n3nc(N)c(=O)[nH]c3=O)cc2Cl)n[nH]c1=O ; [2H]CC(C)c1cc(Oc2c(Cl)cc(-n3nc(N)c(=O)[nH]c3=O)cc2Cl)n[nH]c1=O]

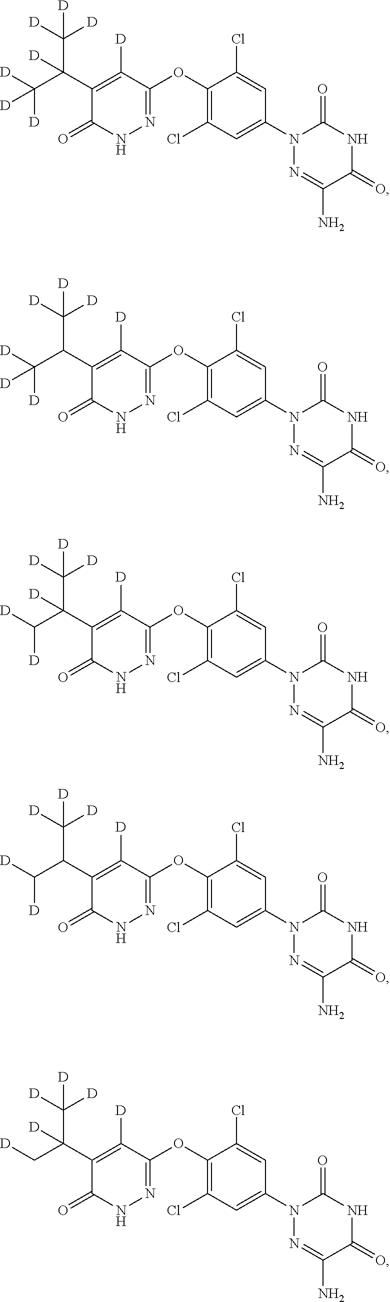 [SMILES: [2H]c1c(Oc2c(Cl)cc(-n3nc(N)c(=O)[nH]c3=O)cc2Cl)n[nH]c(=O)c1C([2H])(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C([2H])(C([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])([2H])[2H] ; [2H]CC([2H])(c1c([2H])c(Oc2c(Cl)cc(-n3nc(N)c(=O)[nH]c3=O)cc2Cl)n[nH]c1=O)C([2H])([2H])[2H]]

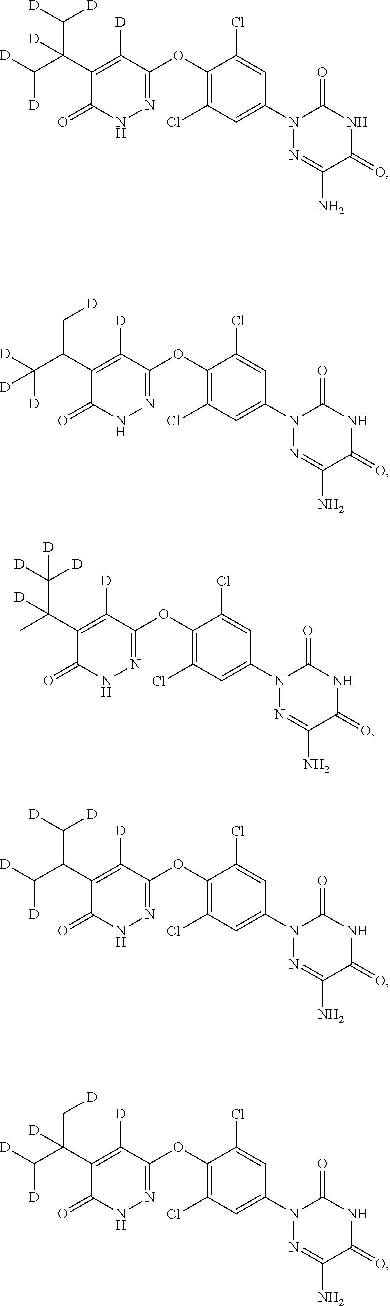 [SMILES: [2H]c1c(Oc2c(Cl)cc(-n3nc(N)c(=O)[nH]c3=O)cc2Cl)n[nH]c(=O)c1C([2H])(C([2H])[2H])C([2H])[2H] ; [2H]CC(c1c([2H])c(Oc2c(Cl)cc(-n3nc(N)c(=O)[nH]c3=O)cc2Cl)n[nH]c1=O)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])[2H] ; [2H]CC([2H])(c1c([2H])c(Oc2c(Cl)cc(-n3nc(N)c(=O)[nH]c3=O)cc2Cl)n[nH]c1=O)C([2H])[2H]]

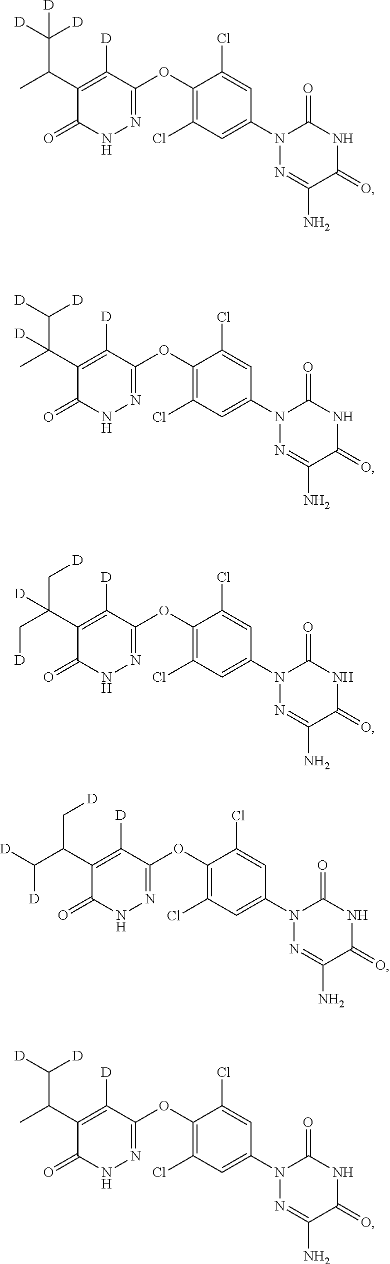 [SMILES: [2H]c1c(Oc2c(Cl)cc(-n3nc(N)c(=O)[nH]c3=O)cc2Cl)n[nH]c(=O)c1C(C)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])[2H] ; [2H]CC([2H])(C[2H])c1c([2H])c(Oc2c(Cl)cc(-n3nc(N)c(=O)[nH]c3=O)cc2Cl)n[nH]c1=O ; [2H]CC(c1c([2H])c(Oc2c(Cl)cc(-n3nc(N)c(=O)[nH]c3=O)cc2Cl)n[nH]c1=O)C([2H])[2H] ; [2H]c1c(Oc2c(Cl)cc(-n3nc(N)c(=O)[nH]c3=O)cc2Cl)n[nH]c(=O)c1C(C)C([2H])[2H]]

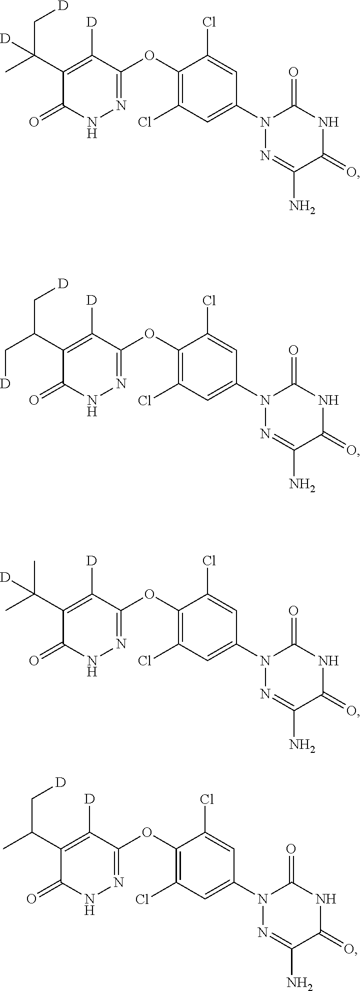 [SMILES: [2H]CC([2H])(C)c1c([2H])c(Oc2c(Cl)cc(-n3nc(N)c(=O)[nH]c3=O)cc2Cl)n[nH]c1=O ; [2H]CC(C[2H])c1c([2H])c(Oc2c(Cl)cc(-n3nc(N)c(=O)[nH]c3=O)cc2Cl)n[nH]c1=O ; [2H]c1c(Oc2c(Cl)cc(-n3nc(N)c(=O)[nH]c3=O)cc2Cl)n[nH]c(=O)c1C([2H])(C)C ; [2H]CC(C)c1c([2H])c(Oc2c(Cl)cc(-n3nc(N)c(=O)[nH]c3=O)cc2Cl)n[nH]c1=O]

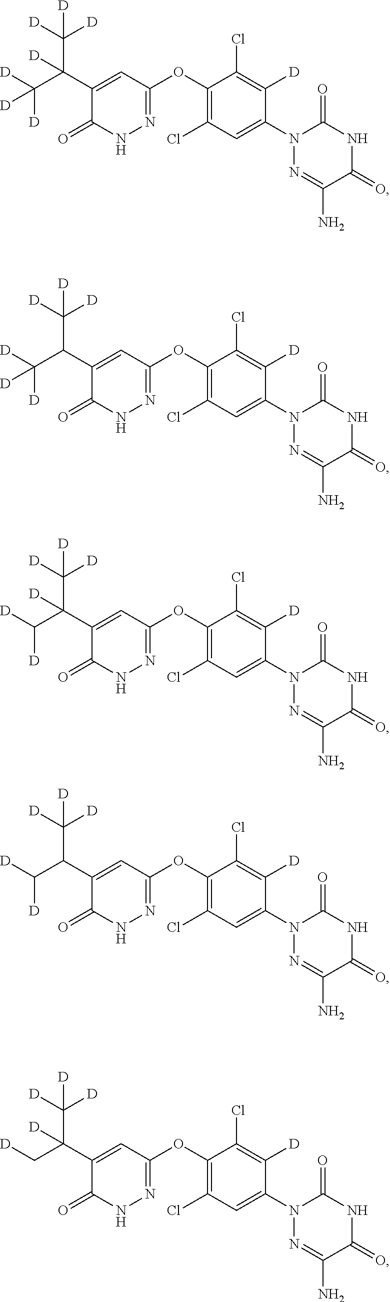 [SMILES: [2H]c1c(-n2nc(N)c(=O)[nH]c2=O)cc(Cl)c(Oc2cc(C([2H])(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C([2H])(C([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])([2H])[2H]]

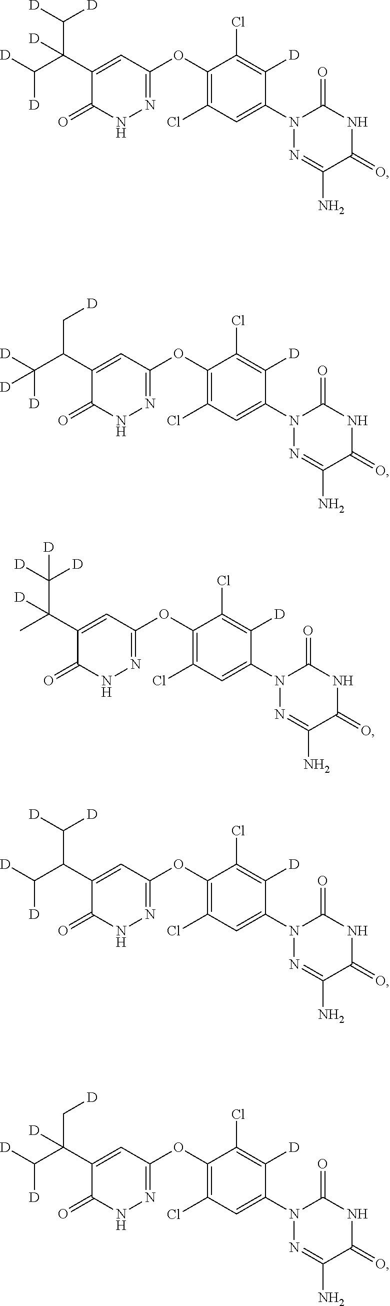 [SMILES: [2H]c1c(-n2nc(N)c(=O)[nH]c2=O)cc(Cl)c(Oc2cc(C([2H])(C([2H])[2H])C([2H])[2H])c(=O)[nH]n2)c1Cl ; [2H]CC(c1cc(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2cc(C([2H])(C)C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])[2H]]

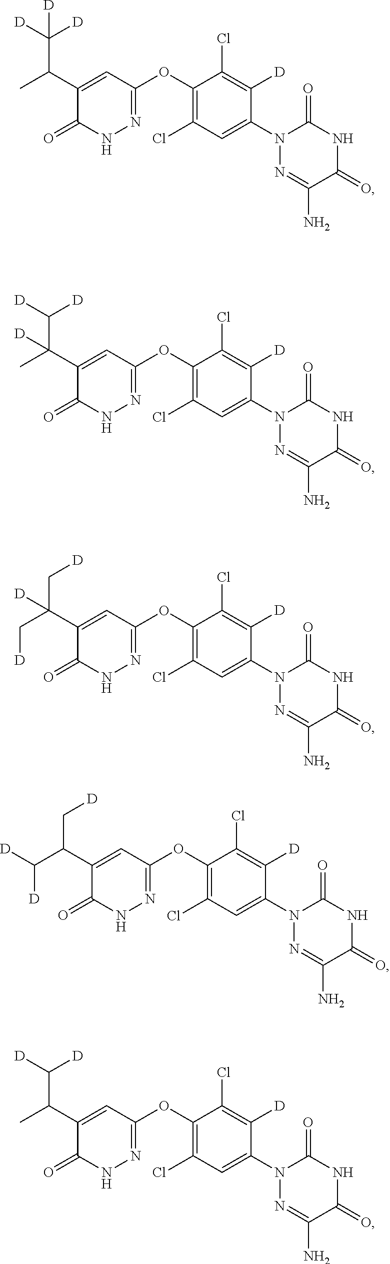 [SMILES: [2H]c1c(-n2nc(N)c(=O)[nH]c2=O)cc(Cl)c(Oc2cc(C(C)C([2H])([2H])[2H])c(=O)[nH]n2)c1Cl ; [2H]c1c(-n2nc(N)c(=O)[nH]c2=O)cc(Cl)c(Oc2cc(C([2H])(C)C([2H])[2H])c(=O)[nH]n2)c1Cl ; [2H]CC([2H])(C[2H])c1cc(Oc2c(Cl)cc(-n3nc(N)c(=O)[nH]c3=O)c([2H])c2Cl)n[nH]c1=O ; [2H]CC(c1cc(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2cc(C(C)C([2H])[2H])c(=O)[nH]n2)c1Cl]

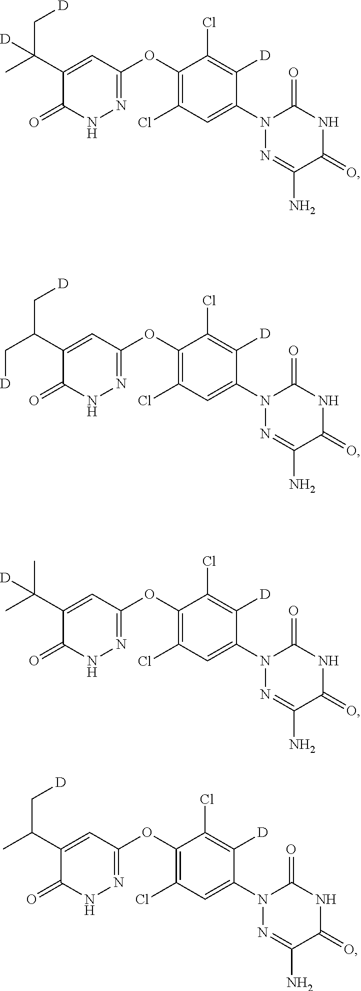 [SMILES: [2H]CC([2H])(C)c1cc(Oc2c(Cl)cc(-n3nc(N)c(=O)[nH]c3=O)c([2H])c2Cl)n[nH]c1=O ; [2H]CC(C[2H])c1cc(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2cc(C([2H])(C)C)c(=O)[nH]n2)c1Cl ; [2H]CC(C)c1cc(Oc2c(Cl)cc(-n3nc(N)c(=O)[nH]c3=O)c([2H])c2Cl)n[nH]c1=O]

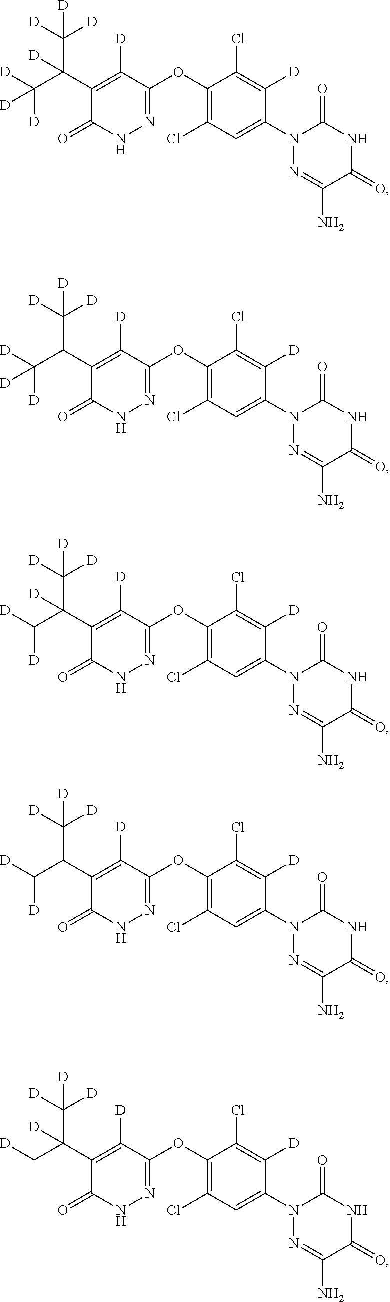

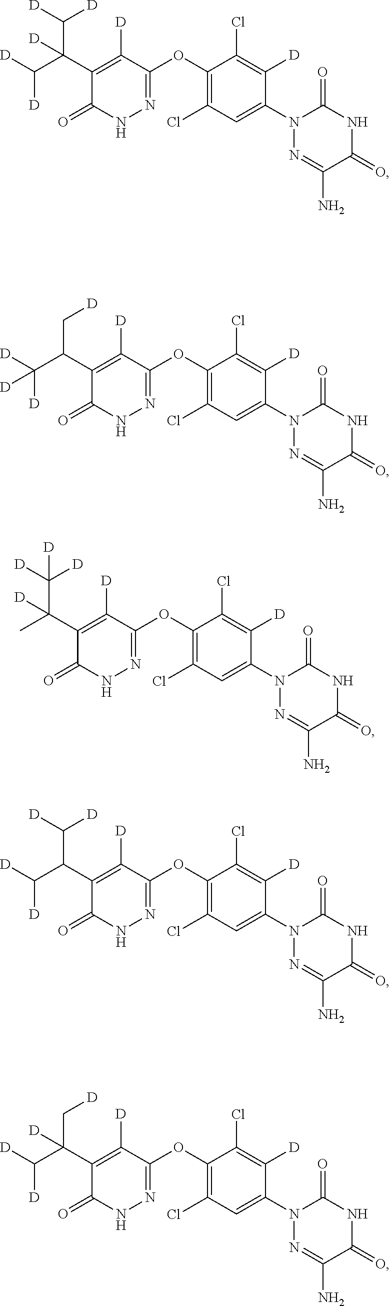 [SMILES: [2H]c1c(-n2nc(N)c(=O)[nH]c2=O)cc(Cl)c(Oc2n[nH]c(=O)c(C([2H])(C([2H])[2H])C([2H])[2H])c2[2H])c1Cl ; [2H]CC(c1c([2H])c(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2n[nH]c(=O)c(C([2H])(C)C([2H])([2H])[2H])c2[2H])c1Cl ; [2H]c1c(-n2nc(N)c(=O)[nH]c2=O)cc(Cl)c(Oc2n[nH]c(=O)c(C(C([2H])[2H])C([2H])[2H])c2[2H])c1Cl ; [2H]CC([2H])(c1c([2H])c(Oc2c(Cl)cc(-n3nc(N)c(=O)[nH]c3=O)c([2H])c2Cl)n[nH]c1=O)C([2H])[2H]]

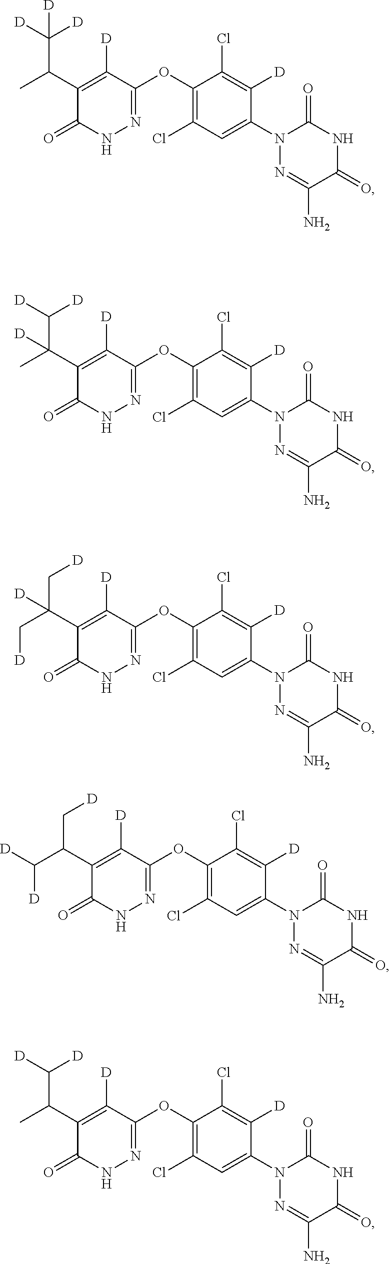 [SMILES: [2H]c1c(-n2nc(N)c(=O)[nH]c2=O)cc(Cl)c(Oc2n[nH]c(=O)c(C(C)C([2H])([2H])[2H])c2[2H])c1Cl ; [2H]c1c(-n2nc(N)c(=O)[nH]c2=O)cc(Cl)c(Oc2n[nH]c(=O)c(C([2H])(C)C([2H])[2H])c2[2H])c1Cl ; [2H]CC([2H])(C[2H])c1c([2H])c(Oc2c(Cl)cc(-n3nc(N)c(=O)[nH]c3=O)c([2H])c2Cl)n[nH]c1=O ; [2H]CC(c1c([2H])c(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2n[nH]c(=O)c(C(C)C([2H])[2H])c2[2H])c1Cl]

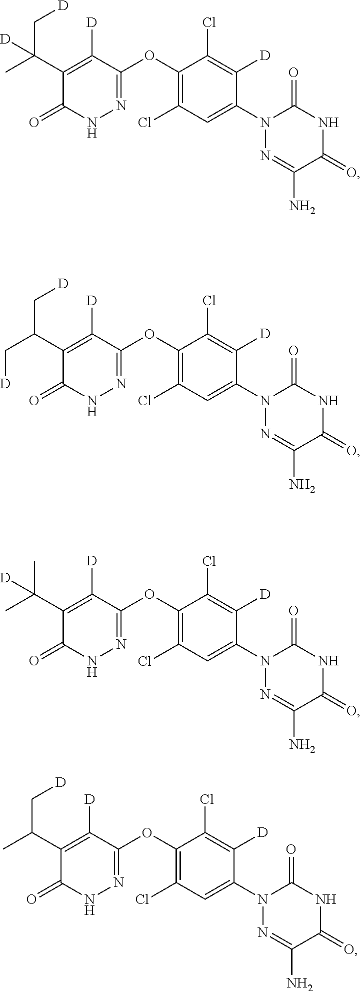 [SMILES: [2H]CC([2H])(C)c1c([2H])c(Oc2c(Cl)cc(-n3nc(N)c(=O)[nH]c3=O)c([2H])c2Cl)n[nH]c1=O ; [2H]CC(C[2H])c1c([2H])c(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2n[nH]c(=O)c(C([2H])(C)C)c2[2H])c1Cl ; [2H]CC(C)c1c([2H])c(Oc2c(Cl)cc(-n3nc(N)c(=O)[nH]c3=O)c([2H])c2Cl)n[nH]c1=O]

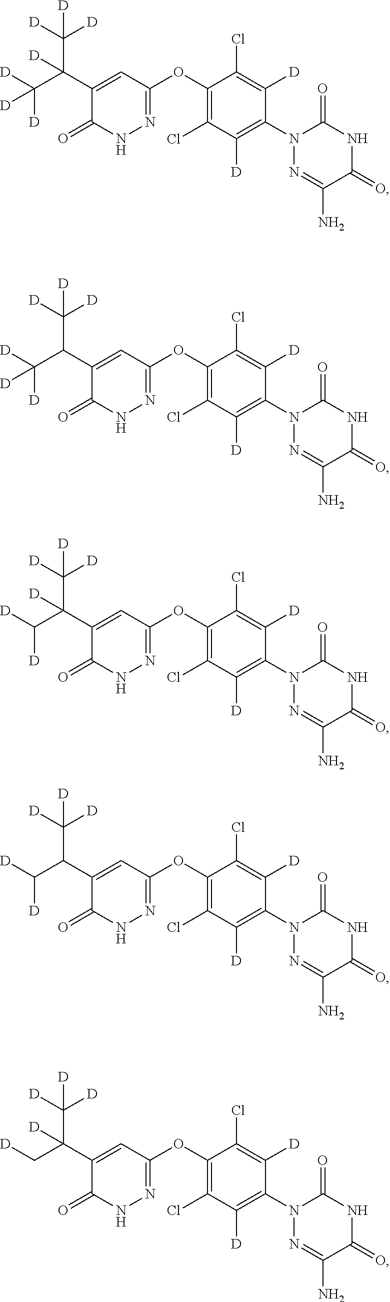

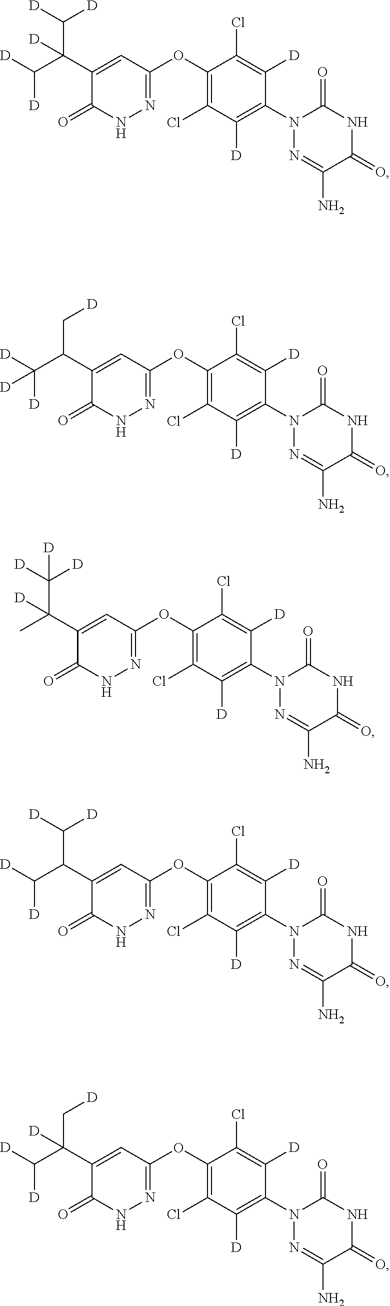 [SMILES: [2H]c1c(Cl)c(Oc2cc(C([2H])(C([2H])[2H])C([2H])[2H])c(=O)[nH]n2)c(Cl)c([2H])c1-n1nc(N)c(=O)[nH]c1=O ; [2H]CC(c1cc(Oc2c(Cl)c([2H])c(-n3nc(N)c(=O)[nH]c3=O)c([2H])c2Cl)n[nH]c1=O)C([2H])([2H])[2H] ; [2H]c1c(Cl)c(Oc2cc(C([2H])(C)C([2H])([2H])[2H])c(=O)[nH]n2)c(Cl)c([2H])c1-n1nc(N)c(=O)[nH]c1=O ; [2H]c1c(Cl)c(Oc2cc(C(C([2H])[2H])C([2H])[2H])c(=O)[nH]n2)c(Cl)c([2H])c1-n1nc(N)c(=O)[nH]c1=O ; [2H]CC([2H])(c1cc(Oc2c(Cl)c([2H])c(-n3nc(N)c(=O)[nH]c3=O)c([2H])c2Cl)n[nH]c1=O)C([2H])[2H]]

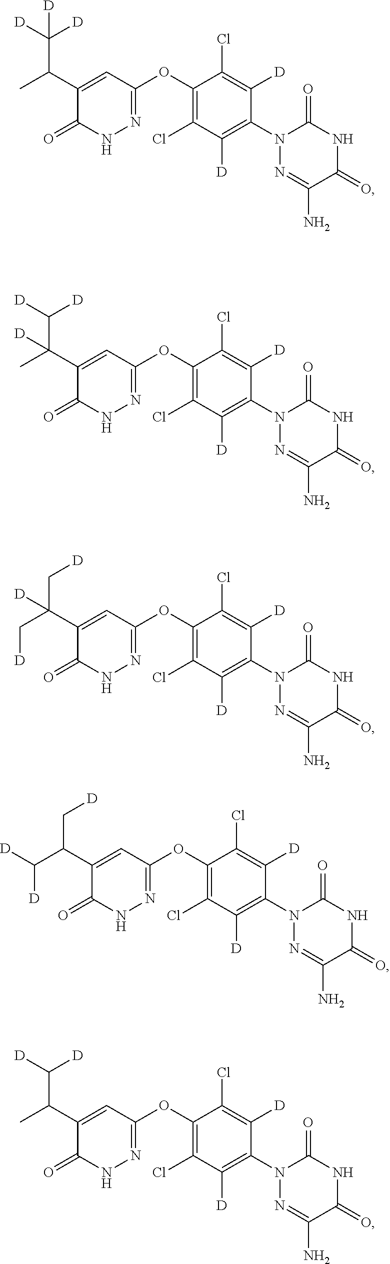 [SMILES: [2H]c1c(Cl)c(Oc2cc(C(C)C([2H])([2H])[2H])c(=O)[nH]n2)c(Cl)c([2H])c1-n1nc(N)c(=O)[nH]c1=O ; [2H]c1c(Cl)c(Oc2cc(C([2H])(C)C([2H])[2H])c(=O)[nH]n2)c(Cl)c([2H])c1-n1nc(N)c(=O)[nH]c1=O ; [2H]CC([2H])(C[2H])c1cc(Oc2c(Cl)c([2H])c(-n3nc(N)c(=O)[nH]c3=O)c([2H])c2Cl)n[nH]c1=O ; [2H]CC(c1cc(Oc2c(Cl)c([2H])c(-n3nc(N)c(=O)[nH]c3=O)c([2H])c2Cl)n[nH]c1=O)C([2H])[2H] ; [2H]c1c(Cl)c(Oc2cc(C(C)C([2H])[2H])c(=O)[nH]n2)c(Cl)c([2H])c1-n1nc(N)c(=O)[nH]c1=O]

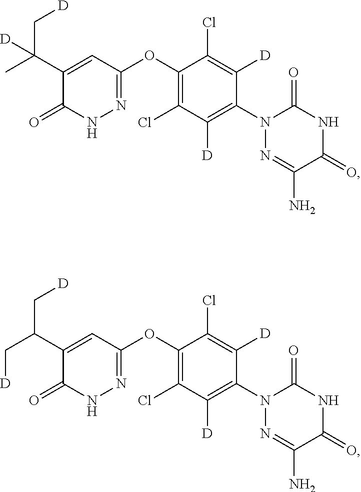 [SMILES: [2H]CC([2H])(C)c1cc(Oc2c(Cl)c([2H])c(-n3nc(N)c(=O)[nH]c3=O)c([2H])c2Cl)n[nH]c1=O ; [2H]CC(C[2H])c1cc(Oc2c(Cl)c([2H])c(-n3nc(N)c(=O)[nH]c3=O)c([2H])c2Cl)n[nH]c1=O]

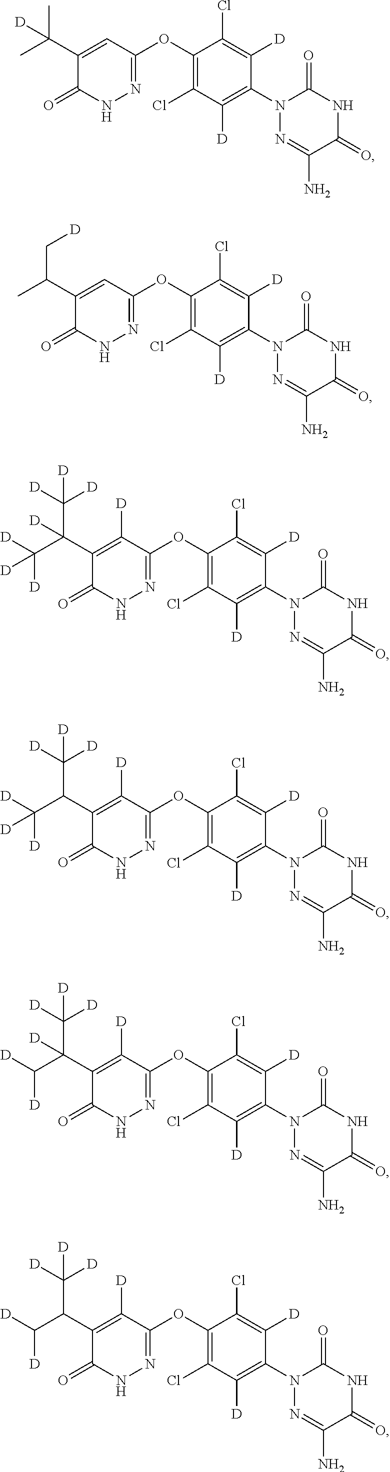

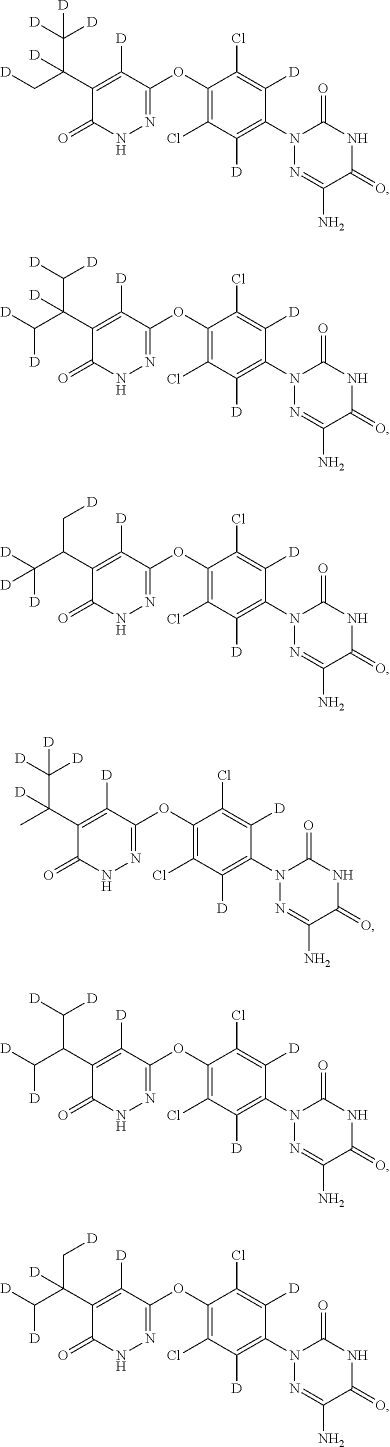

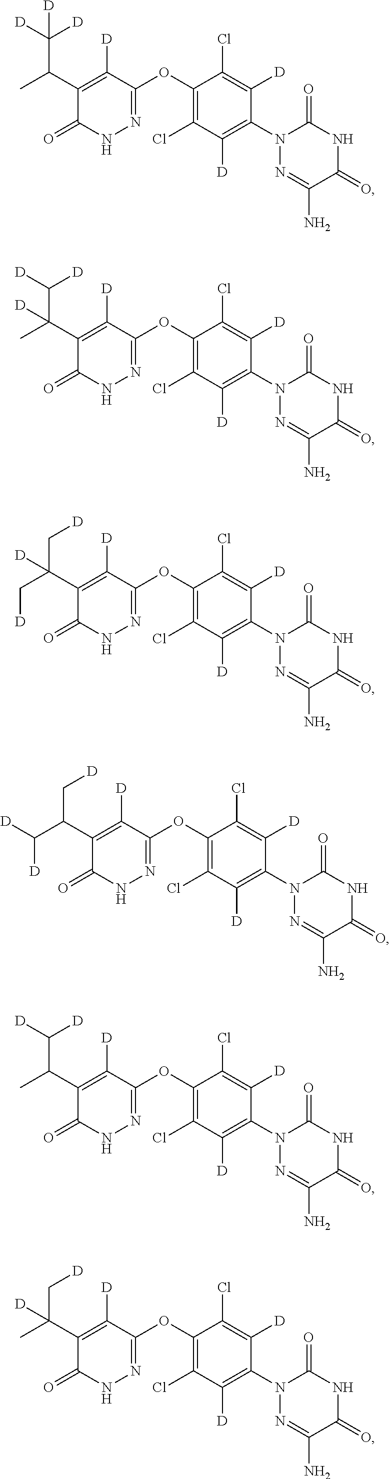

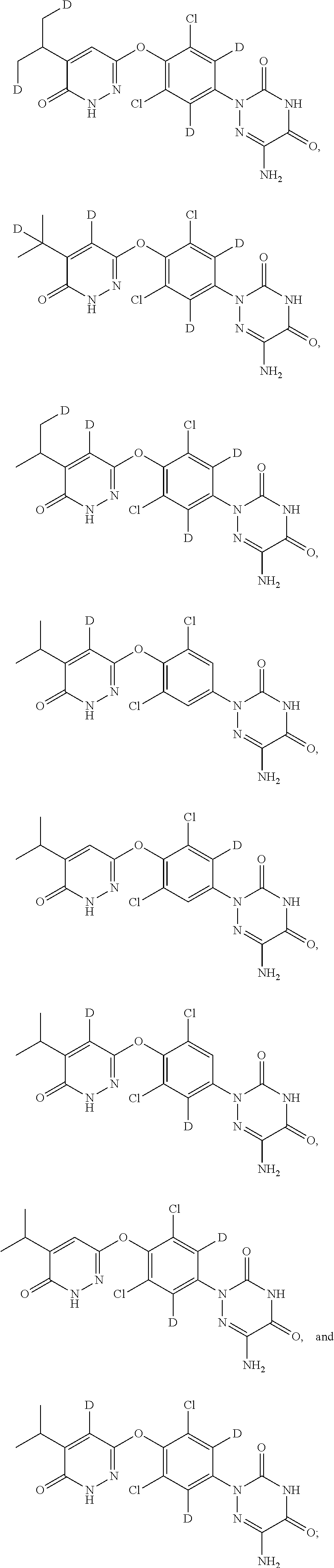

or a stereoisomer or a tautomer thereof, or a pharmaceutically acceptable salt of the compound, the stereoisomer, or the tautomer. In some embodiments, the compound is

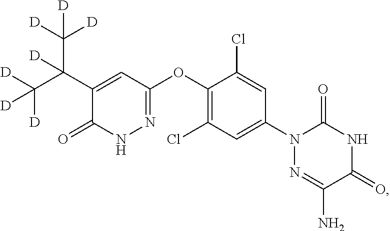 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H]]

or a stereoisomer or a tautomer thereof, or a pharmaceutically acceptable salt of the compound, the stereoisomer, or the tautomer.

### Pharmaceutical Compositions

In another aspect, disclosed herein are pharmaceutical compositions comprising, consisting essentially of, or consisting of a compound as described herein, and at least one pharmaceutically acceptable excipient.

In another aspect, disclosed herein are pharmaceutical compositions comprising, consisting essentially of, or consisting of a compound of Formula I, as described herein, and at least one pharmaceutically acceptable excipient.

The pharmaceutical composition disclosed herein may comprise a pharmaceutically acceptable carrier, such as diluents, disintegrants, sweetening agents, glidants, or flavoring agents and may be formulated into an oral dosage form such as tablets, capsules, powders, granules, suspensions, emulsions, or syrups; or a parenteral dosage form such as liquids for external use, suspensions for external use, emulsions for external use, gels (ointments or the like), inhaling agents, spraying agents, injections, etc. Said dosage forms may be formulated in various forms, e.g., a dosage form for single administration or for multiple administrations.

The pharmaceutical composition disclosed herein may include excipients such as lactose, corn starch, or the like, glidants such as magnesium stearate, etc., emulsifying agents, suspending agents, stabilizers, and isotonic agents, etc. If desired, a sweetening agent and/or a flavoring agent may be added. Exemplary excipients include, without limitation, polyethylene glycol (PEG), hydrogenated castor oil (HCO), cremophors, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Inorganic salt or buffers include, but are not limited to, citric acid, sodium chloride, potassium chloride, sodium sulfate, potassium nitrate, sodium phosphate monobasic, sodium phosphate dibasic, and combinations thereof.

Suitable antioxidants for use in the present disclosure include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional exemplary excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition disclosed herein may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Non-limiting examples of suitable bases include bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Generally, however, the excipient will be present in the composition in an amount of about 1% to about 99% by weight, preferably from about 5% to about 98% by weight, more preferably from about 15 to about 95% by weight of the excipient. In general, the amount of excipient present in a composition of the disclosure is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, transdermal, rectal, transmucosal, or intestinal administration; parenteral delivery, including intramuscular, subcutaneous, intravenous, intramedullary injections, as well as inhalation, intrathecal, direct intraventricular, intraperitoneal, intranasal, or intraocular injections.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. These pharmaceutical compositions, then, may be formulated in a conventional manner using one or more known physiologically acceptable carriers comprising excipients and/or auxiliaries, which facilitate processing of the active compounds into preparations that can be used pharmaceutically. Any of the well-known techniques, carriers, and excipients may be used as suitable and as understood in the art; e.g., in Remington's Pharmaceutical Sciences, above.

Pharmaceutical compositions suitable for use in the presently disclosed formulations include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. In some embodiments, a therapeutically effective amount means an amount of compound effective to alleviate or ameliorate symptoms of disease or prolong the survival of the subject being treated.

Although the exact dosage can be determined on a drug-by-drug basis, in most cases, some generalizations regarding the dosage can be made. The daily dosage regimen for an adult human patient may be, for example, an oral dose of between 0.001 mg and 1000 mg of each ingredient, preferably between 0.01 mg and 500 mg, for example 1 to 200 mg or each active ingredient of the pharmaceutical compositions disclosed herein or a pharmaceutically acceptable salt thereof calculated as the free base or free acid, the composition being administered 1 to 4 times per day or per week. Alternatively, the compositions disclosed herein may be administered by continuous such as sustained, delayed, or extended release, preferably at a dose of each ingredient up to 500 mg per day. Thus, the total daily dosage by oral administration of each ingredient will typically be in the range 0.1 mg to 2000 mg.

### Methods of Treatment

In another aspect, disclosed herein are methods of treating a thyroid hormone receptor related disorder in a patient, the method comprising, consisting essentially of, or consisting of the steps of identifying a patient in need of treatment for the thyroid hormone receptor related disorder, and administering to the patient, or contacting the patient with, a compound as described herein.

In another aspect, disclosed herein are methods of treating a thyroid hormone receptor related disorder in a patient, the method comprising, consisting essentially of, or consisting of the steps of identifying a patient in need of treatment for the thyroid hormone receptor related disorder, and administering to the patient, or contacting the patient with, a compound of Formula I, as described herein.

In some embodiments, a health care professional, such as a physician, physician's assistant, nurse practitioner, or the like, identifies an individual as being in need of treatment for the thyroid hormone receptor related disorder, and/or a candidate for treatment with a compound disclosed herein. The identification may be based on medical test results, non-responsiveness to other, first-line therapies, the specific nature of the particular liver disorder, or the like.

In some embodiments, the thyroid hormone receptor related disorder is selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

In another aspect, disclosed herein are methods of treating a disorder or disease in a subject in need thereof, the method comprising, consisting essentially of, or consisting of administering to the subject a therapeutically effective amount of a compound or composition disclosed herein, wherein the disorder or disease is selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

In another aspect, disclosed herein are methods of treating MASH in a subject in need thereof, the method comprising, consisting essentially of, or consisting of administering to the subject a therapeutically effective amount of a compound or composition disclosed herein.

In another aspect, disclosed herein are methods of treating obesity in a subject in need thereof, the method comprising, consisting essentially of, or consisting of administering to the subject a therapeutically effective amount of a compound or composition disclosed herein.

In another aspect, disclosed herein are methods of treating hyperlipidemia in a subject in need thereof, the method comprising, consisting essentially of, or consisting of administering to the subject a therapeutically effective amount of a compound or composition disclosed herein.

In another aspect, disclosed herein are methods of treating hypercholesterolemia in a subject in need thereof, the method comprising, consisting essentially of, or consisting of administering to the subject a therapeutically effective amount of a compound or composition disclosed herein.

In another aspect, disclosed herein are methods of treating diabetes in a subject in need thereof, the method comprising, consisting essentially of, or consisting of administering to the subject a therapeutically effective amount of a compound or composition disclosed herein.

In another aspect, disclosed herein are methods of treating liver steatosis in a subject in need thereof, the method comprising, consisting essentially of, or consisting of administering to the subject a therapeutically effective amount of a compound or composition disclosed herein.

In some embodiments, the compound as described herein, or the stereoisomer or the tautomer thereof, or the pharmaceutically acceptable salt thereof, or the pharmaceutical composition described herein, is administered in combination with a KHK inhibitor, an FXR agonist, a SSAO inhibitor, a FASN inhibitor, or a SCD1 modulator. In some embodiments, the KHK inhibitor is PF-06835919; the FXR agonist is TERN-101 (LY2562175), tropifexor, obeticholic acid (OCA), or ASC42; the SSAO inhibitor is TERN-201; the FASN inhibitor is ASC40; and the SCD1 modulator is aramchol.

In another aspect, disclosed herein are methods of selectively modulating the activity of a thyroid hormone receptor beta (THR-β) comprising, consisting essentially of, or consisting of contacting a compound as described herein, with a thyroid hormone receptor.

In another aspect, disclosed herein are methods of selectively modulating the activity of a thyroid hormone receptor beta (THR-β) comprising, consisting essentially of, or consisting of contacting a compound of Formula I, as described herein, with a thyroid hormone receptor.

In some embodiments, the contacting is in vitro or ex vivo. In some embodiments, the contacting is in vivo.

In another aspect, disclosed herein are methods of selectively modulating the activity of a thyroid hormone receptor beta (THR-β) comprising, consisting essentially of, or consisting of contacting a compound of Formula I, as described herein, with a thyroid hormone receptor in a patient suffering from a thyroid hormone receptor related disorder selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

### EXAMPLES

### Example 1. 6-amino-2-(3,5-dichloro-4-((6-oxo-5-(propan-2-yl-d₇)-1,6-dihydropyridazin-3-yl)oxy)phenyl)-1,2,4-triazine-3,5-(2H, 4H)-dione (Compound 1)

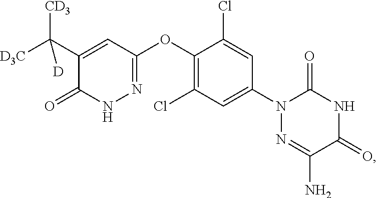 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H]]

### Step A. Synthesis of 3,6-dichloro-4-(propan-2-yl-d₇)pyridazine

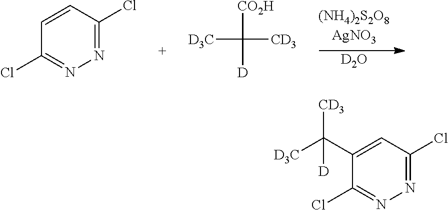

A mixture containing 3,6-dichloropyridazine (1.57 g, 148.97 g/mol, 1.0 eq), silver nitrate (0.35 g, 169.87 g/mol, 0.2 eq), and deuterium oxide (26 mL) was stirred under Argon. 2-(Methyl-d₃)propanoic-2,3,3,3-d₄ acid (1.00 g, 95.15 g/mol, 1.0 eq) was added and the resulting solution heated to 40° C. A solution of ammonium persulfate (2.40 g, 228.20 g/mol, 1.0 eq) in deuterium oxide (7 mL) was added so that the internal temperature was maintained at 40-45° C. The resulting solution was maintained at 40° C. for 1 hour then cooled and filtered. The filtrate was extracted with dichloromethane then the extract was filtered through celite and sodium sulfate. The filtrate was concentrated to a colorless oil (2.17 g) which was dissolved in hexane (6 mL) and purified by normal phase silica gel chromatography using a linear gradient of 0 to 30% ethyl acetate in hexane over 12 column volumes. Fractions containing product were concentrated to a colorless oil (1.84 g). LCMS (ESI, m/z): 198 [M+H]⁺.

### Step B. Synthesis of 3,5-dichloro-4-((6-chloro-5-(propan-2-yl-d₇)pyridazin-3-yl)oxy)aniline

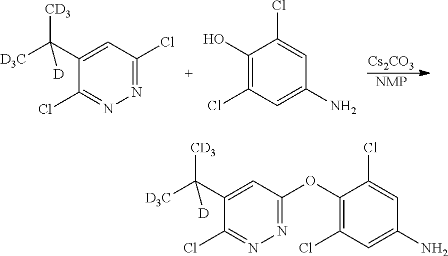

3,6-Dichloro-4-(propan-2-yl-d₇)pyridazine (1.84 g, 198.10 g/mol, 1.0 eq), 4-amino-2,6-dichlorophenol (1.65 g, 178.01 g/mol, 1.0 eq), cesium carbonate (3.48 g, 325.82 g/mol, 1.2 eq) and N-methyl-2-pyrrolidone (NMP) (9 mL) were combined, stirred, and heated under Argon at 70° C. for 19 hours. NMP (12 mL) was added. The solution was cooled with a room temperature water bath while water (21 mL) was dripped in. The resulting slurry was stirred for one hour and filtered. The filter cake was washed with 1:1 NMP:water then with water. The filter cake was recrystallized by adding isopropanol (25 mL), heating to 73° C., and cooling. Fifteen minutes after recrystallization commenced, water (22 mL) was dripped in. The resulting slurry was stirred for 0.5 hour, cooled using an ice-water bath, and filtered. The filter cake was washed with 1:1 isopropanol:water then dried at 70° C. under vacuum to provide solid product (1.02 g).

### Step C. Synthesis of N-(3,5-dichloro-4-((6-oxo-5-(propan-2-yl-d₇)-1,6-dihydropyridazin-3-yl)oxy)acetamide

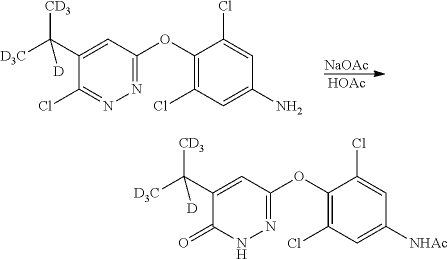

3,5-Dichloro-4-((6-chloro-5-(propan-2-yl-d₇)pyridazin-3-yl)oxy)aniline (1.02 g, 339.65 g/mol, 1.0 eq), sodium acetate (0.87 g, 82.03 g/mol, 3.5 eq), and acetic acid (10 mL) were combined, stirred and heated in a 115° C.-heating mantle for 17 hours. The mixture was cooled to room temperature and mixed with water to generate solids. These were collected but were found to be impure mixtures because of incomplete reaction. So, they were re-exposed to the same reaction conditions for another 24 hours. At that time, they were cooled, diluted with water, and filtered. The filter cake was washed with 2:1 water:acetic acid and dried in a vacuum oven at 60° C. to provide desired product (0.56 g). LCMS (ESI, m/z): 363 [M+H]⁺.

### Step D. Synthesis of 6-(4-amino-2,6-dichlorophenoxy)-4-(propan-2-yl-d7)pyridazine-3(2H)-one

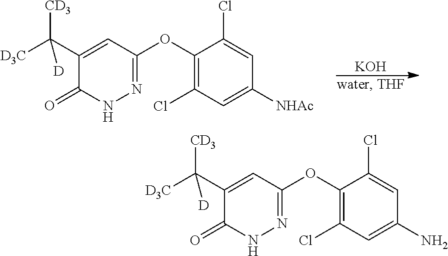

N-(3,5-Dichloro-4-((6-oxo-5-(propan-2-yl-d₇)-1,6-dihydropyridazin-3-yl)oxy)acetamide (0.56 g, 363.25 g/mol, 1.0 eq), potassium hydroxide (0.45 g, 56.11 g/mol, 5.2 eq), water (3.5 mL), and tetrahydrofuran (1 mL) were heated in a sealed vial at 64° C. for 2 days. The resulting slurry was cooled, concentrated partially to remove tetrahydrofuran, and filtered. The filter cake was washed with water and dried in a 65° C.-vacuum oven, providing pink solid product (0.55 g). LCMS (ESI, m/z): 321 [M+H]⁺.

### Step E. Synthesis of ethyl (Z)-2-(2-(3,5-dichloro-4-((6-oxo-5-(propan-2-yl-d₇)-1,6-dihydropyridazin-3-yl)oxy)phenyl)hydrazineylidene)-3-(((ethoxycarbonyl)amino)-3-oxopropanoate

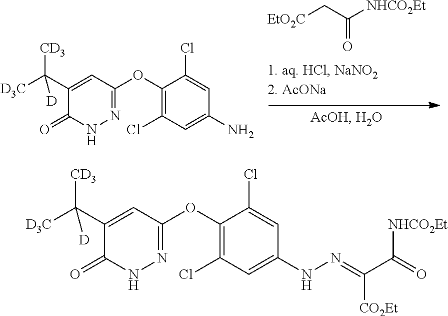

6-(4-Amino-2,6-dichlorophenoxy)-4-(propan-2-yl-d₇)pyridazine-3(2H)-one (0.54 g, 321.21 g/mol, 1.00 eq), ethyl 3-((ethoxycarbonyl)amino)-3-oxopropanoate (0.35 g, 203.19 g/mol, 1.05 eq), acetic acid (19 mL), and water (7 mL) were combined, stirred, and cooled using an ice-water bath. Between 5-10° C., concentrated hydrochloric acid (0.50 g, 12 M, 3 eq) was added followed by drop-wise addition of a solution of sodium nitrite (118 mg, 69 g/mol, 1.03 eq) in water (0.2 mL). Cooling was maintained for 50 minutes then sodium acetate (0.42 g, 82.03 g/mol, 3.08 eq) was added. Cooling was maintained for 50 minutes then the ice-water bath was replaced with one containing 25° C.-water. After 55 minutes, the reaction was filtered and the filter cake was washed with 2:1 AcOH:water and then with water. It was dried in a 60° C.-vacuum oven to provide orange solid product (0.56 g). LCMS (ESI, m/z): 535 [M+H]⁺.

### Step F. Synthesis of ethyl 2-(3,5-dichloro-4-((6-oxo-5-(propan-2-yl-d₇)-1,6-dihydropyridazin-3-yl)oxy)phenyl)-3,5-dioxo-2,3,4,5-tetrahydro-1,2,4-triazine-6-carboxylate

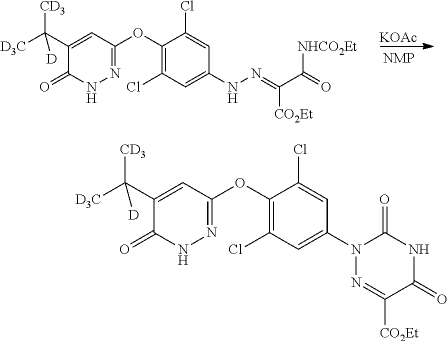

Ethyl (Z)-2-(2-(3,5-dichloro-4-((6-oxo-5-(propan-2-yl-d₇)-1,6-dihydropyridazin-3-yl)oxy)phenyl)hydrazineylidene)-3-(((ethoxycarbonyl)amino)-3-oxopropanoate (553 mg, 535.39 g/mol, 1.0 eq), potassium acetate (111 mg, 98.14 g/mol, 1.1 eq), and NMP (5 mL) were combined under Argon, stirred, and heated to 110° C. for 1 hour. The mixture was cooled to room temperature, and water added dropwise until crystallization commenced. After 15 minutes, more water was added dropwise until the ratio of NMP to water was 1:1. After stirring for 5 more minutes, the slurry was filtered, and the filter cake was washed with 2:1 water:NMP followed by washing with water. The collected solids were dried in a 70° C.-vacuum oven to provide desired product (486 mg). LCMS (ESI, m/z): 489 [M+H]⁺.

### Step G. Synthesis of 2-(3,5-dichloro-4-((6-oxo-5(propan-2-yl-d₇)-1,6-dihydropyridazin-3-yl)oxy)phenyl)-3,5-dioxo-2,3,4,5-tetrahydro-1,2,4-triazine-6-carboxylic acid

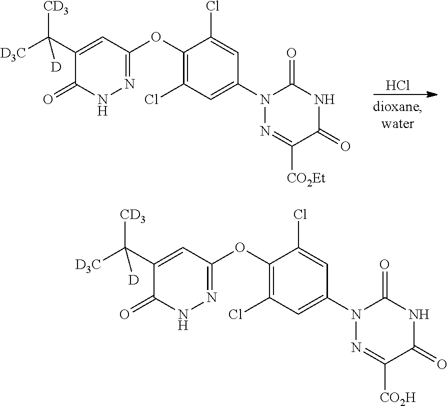

Ethyl 2-(3,5-dichloro-4-((6-oxo-5-(propan-2-yl-d₇)-1,6-dihydropyridazin-3-yl)oxy)phenyl)-3,5-dioxo-2,3,4,5-tetrahydro-1,2,4-triazine-6-carboxylate (486 mg) was mixed with 1,4-dioxane (2 mL) and concentrated hydrochloric acid (5 mL). The mixture was stirred under Argon and heated in a 130° C.-heating block for 1.5 hours. The mixture was then cooled and filtered. The collected solids were washed with 1,4-dioxane and dried at 70° C. in a vacuum oven to provide desired product (366 mg). LCMS (ESI, m/z): 461 [M+H]+.

### Step H. Synthesis of tert-butyl (2-(3,5-dichloro-4-((6-oxo-5-(propan-2-yl-d₇)-1,6-dihydropyridazin-3-yl)oxy)phenyl)-3,5-dioxo-2,3,4,5-tetrahydro-1,2,4-triazin-6-yl)carbamate

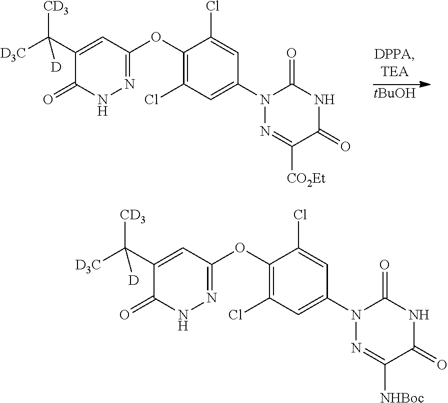

2-(3,5-Dichloro-4-((6-oxo-5(propan-2-yl-d)-1,6-dihydropyridazin-3-yl)oxy)phenyl)-3,5-dioxo-2,3,4,5-tetrahydro-1,2,4-triazine-6-carboxylic acid (363 mg, 461.26 g/mol, 1.0 eq) and t-butanol (1.4 mL) were combined under Argon and stirred. Triethylamine (239 mg, 101.19 g/mol, 3.0 eq) was added followed by diphenylphosphoryl azide (325 mg, 275.2 g/mol, 1.5 eq). The solution was heated to 80° C. for 20 hours then cooled. Citric acid (2 mL, 10% aq. solution) was added dropwise. The resulting slurry was stirred for 1.5 hours and then filtered. The collected solids were washed with a solution containing equal volumes of t-butanol and 10% aq. citric acid. The wet filter cake was mixed with dimethylformamide (3 mL) and heated to 40° C. to dissolve all solids. The solution was cooled to room temperature and water (1 mL) was added dropwise to initiate crystallization. After 17 minutes, the resulting slurry was cooled in an ice-water bath for 15 minutes, filtered, washed with 1:1 DMF:water, and washed with water. The filter cake was dried in a 65° C.-vacuum oven to provide desired product (296 mg). LCMS (ESI, m/z): 532 [M+H]⁺.

### Step I. Synthesis of 6-amino-2-(3,5-dichloro-4-((6-oxo-5-(propan-2-yl-dr)-1,6-dihydropyridazin-3-yl)oxy)phenyl)-1,2,4-triazine-3,5-(2H, 4H)-dione

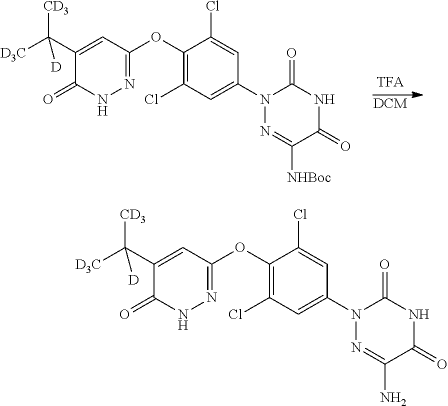

tert-Butyl (2-(3,5-dichloro-4-((6-oxo-5-(propan-2-yl-d₇)-1,6-dihydropyridazin-3-yl)oxy)phenyl)-3,5-dioxo-2,3,4,5-tetrahydro-1,2,4-triazin-6-yl)carbamate (296 mg) was mixed with dichloromethane (7 mL). Trifluoroacetic acid (2 mL) was added and the mixture stirred for 16 hours. It was concentrated, mixed with ethyl acetate (35 mL), and washed with saturated aqueous sodium hydrogen carbonate (3×). The washes were combined and extracted with ethyl acetate. The organic phases were combined, washed with brine, and filtered through sodium sulfate. The filtrate was concentrated and then re-dissolved in NMP (1 mL), stirred, before adding water (1 mL) dropwise to initiate crystallization. After 0.5 hour, the solids were filtered and washed with 2:1 water:NMP then with water. The filter cake was dried in a vacuum oven at 65° C. to provide a solid material (128 mg). This solid material was dissolved in DMF, combined with silica gel (2.7 g), concentrated, and subjected to normal phase chromatography using a 0-to-20% linear gradient of methanol-in-dichloromethane over 10 column volumes. The fractions containing product were concentrated and recrystallized from acetone (50 mL). The solid material was dried at 60° C. in a vacuum oven to afford desired product (66 mg). LCMS (ESI, m/z): 432 [M+H]⁺. ¹H NMR (400 MHz, DMSO-d₆) δ ppm: 12.26 (br s, 1H), 12.18 (s, 1H), 7.85 (s, 2H), 7.41 (s, 1H), 6.45 (s, 2H).

### Example 2. Comparison of Compound 1 with Non-Deuterated Analog (Compound A)

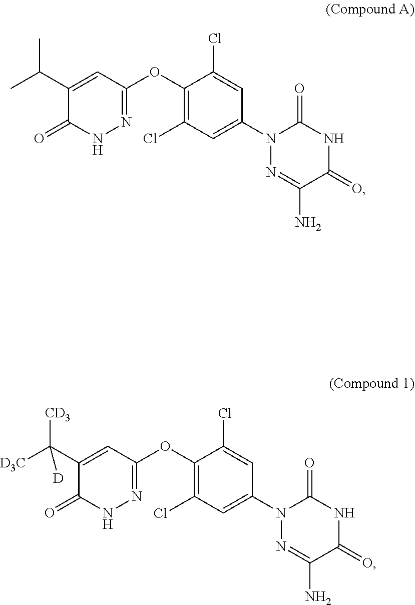 [SMILES: CC(C)c1cc(Oc2c(Cl)cc(-n3nc(N)c(=O)[nH]c3=O)cc2Cl)n[nH]c1=O ; [2H]C([2H])([2H])C([2H])(c1cc(Oc2c(Cl)cc(-n3nc(N)c(=O)[nH]c3=O)cc2Cl)n[nH]c1=O)C([2H])([2H])[2H]]

Compound A and compound 1 exhibited similar in vitro activity profiles with respect to one another when tested against THR-α and THR-β. Both compounds were metabolically stable in human liver microsomes and had high metabolic stability similar to one another in mouse liver microsomes.

However, compound A and compound 1 exhibited differences in rat hepatocytes. Compound A and compound 1 (10 μM for each) were incubated with rat hepatocytes at 37° C. for 120 min. The positive control, 7-ethoxycoumarin (7-EC) at 30 μM, was run concurrently to assess Phase I and Phase II metabolic activities in hepatocytes. The samples were analyzed by LC-UV-MS.

The major metabolite of Compound A resulted from mono-oxygenation of the isopropyl group and occurred at a relative abundance of 9.87% (UV peak area, % total). In contrast, the analogous mono-oxygenated metabolite formation was 2.45% for Compound 1. Moreover, Compound 1 was more stable (95% remaining) compared to Compound A (90% remaining) in rat hepatocytes.

### REFERENCES

1. Younossi, Z M, Koenig, A B, Abdelatif, D, Fazel, Y, Henry, L, Wymer, M. Global epidemiology of nonalcoholic fatty liver disease-Meta-analytic assessment of prevalence, incidence, and outcomes. Hepatology, 2016, 64(1):73e84.
2. Gastroenterology. 2012 June; 142(7): 1592-609. doi: 10.1053/j.gastro.2012.04.001. Epub 2012 May 15.
3. Serfaty, L., Lemoine, M. Definition and natural history of metabolic steatosis: clinical aspects of NAFLD, NASH and cirrhosis. Diabetes and Metabolism, 2008, 34 (6 Pt 2):634e637.
4. Hepatology. 2012 October; 56(4): 1580-1584. doi: 10.1002/hep.26031
5. Dulai, P S, Singh, S, Patel, J, Soni, M, Prokop, L J, Younossi, Z, et al., Increased risk of mortality by fibrosis stage in nonalcoholic fatty liver disease: systematic review and meta-analysis. Hepatology, 2017, 65(5):1557e1565.
6. Younossi, Z M, Loomba, R, Rinella, M E, Bugianesi, E, Marchesini, G, Neuschwander-Tetri, B A, et al. Current and future therapeutic regimens for non-alcoholic fatty liver disease (NAFLD) and non-alcoholic steatohepatitis (NASH). Hepatology, 2018, 68(1):349e360.
7. Harvey C B, Williams G R. Mechanism of thyroid hormone action. Thyroid, 2002 June; 12(6):441-6.
8. Bookout A L, Jeong Y, Downes M, Yu R T, Evans R M, Mangelsdorf D J. Anatomical profiling of nuclear receptor expression reveals a hierarchical transcriptional network. Cell, 2006, 126:789-799
9. Flamant F, Baxter J D, Forrest D, Refetoff S, Samuels H H, Scanlan T S, Vennstrom B, Samarut J. International union of pharmacology. LIX. The pharmacology and classification of the nuclear receptor superfamily: thyroid hormone receptors. Pharmacol. Rev., 2006, 58:705-711
10. Haning H, Woltering M, Mueller U, Schmidt G, Schmeck C, Voehringer V, Kretschmer A, Pernerstorfer J. Bioorg. Med Chem Lett., 2005 Apr. 1, 15(7): 1835-40. Novel heterocyclic thyromimetics.
11. Hirano T, Kagechika H. Thyromimetics: a review of recent reports and patents (2004-2009). Expert Opin Ther Pat., 2010 February; 20(2):213-28. doi: 10.1517/13543770903567069.
12. Kowalik M A, Columbano A, Perra A. Thyroid Hormones, Thyromimetics and Their Metabolites in the Treatment of Liver Disease. Front Endocrinol (Lausanne), 2018 Jul. 10; 9:382. doi: 10.3389/fendo.2018.00382. eCollection 2018.
13. Hartley M D, Kirkemo L L, Banerji T, Scanlan T S. A Thyroid Hormone-Based Strategy for Correcting the Biochemical Abnormality in X-Linked Adrenoleukodystrophy. Endocrinology 2017, 158(5), p 1328-1338. doi: 10.1210/en.2016-1842.
14. Milanesi A, Brent G A. Beam Me In: Thyroid Hormone Analog Targets Alternative Transporter in Mouse Model of X-Linked Adrenoleukodystrophy. Endocrinology 2017, 158, p 1116-1119. doi: 10.1210/en.2017-00206.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A compound of Formula I: 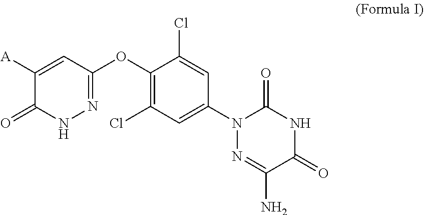
or a stereoisomer or a tautomer thereof; or a pharmaceutically acceptable salt of the compound, the stereoisomer, or the tautomer; wherein A is an isopropyl group, and 1 to 7 hydrogen atoms on the isopropyl group are replaced with deuterium atom(s).

2. The compound, stereoisomer, tautomer, or pharmaceutically acceptable salt of claim 1, wherein all 7 hydrogen atoms on the isopropyl group are replaced with deuterium atoms.

3. The compound, stereoisomer, tautomer, or pharmaceutically acceptable salt of claim 1, wherein 1 to 6 hydrogen atoms on the isopropyl group are replaced with deuterium atoms.

4. The compound, stereoisomer, tautomer, or pharmaceutically acceptable salt of claim 1, wherein 1 to 4 hydrogen atoms on the isopropyl group are replaced with deuterium atoms.

5. A compound selected from the group consisting of: 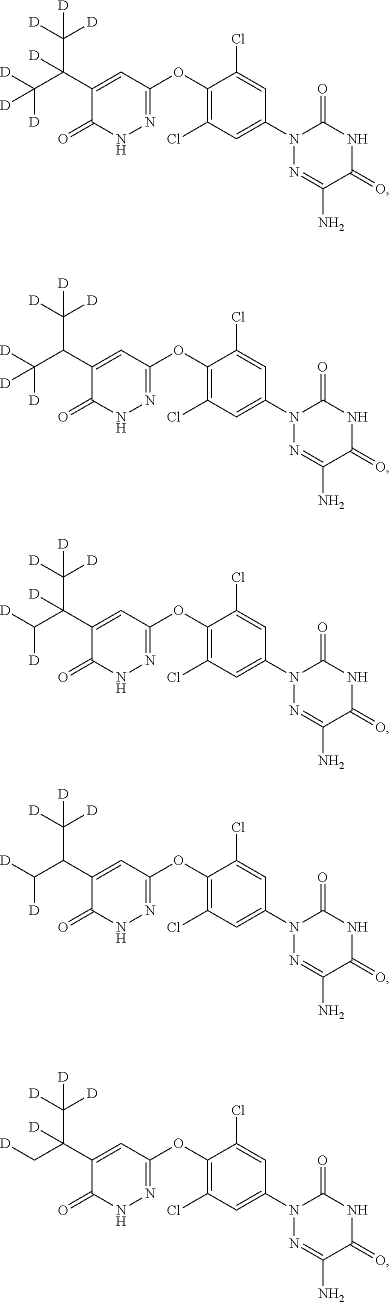 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])([2H])C(c1cc(Oc2c(Cl)cc(-n3nc(N)c(=O)[nH]c3=O)cc2Cl)n[nH]c1=O)C([2H])([2H])[2H] ; [2H]C([2H])C([2H])(c1cc(Oc2c(Cl)cc(-n3nc(N)c(=O)[nH]c3=O)cc2Cl)n[nH]c1=O)C([2H])([2H])[2H] ; [2H]C([2H])C(c1cc(Oc2c(Cl)cc(-n3nc(N)c(=O)[nH]c3=O)cc2Cl)n[nH]c1=O)C([2H])([2H])[2H] ; [2H]CC([2H])(c1cc(Oc2c(Cl)cc(-n3nc(N)c(=O)[nH]c3=O)cc2Cl)n[nH]c1=O)C([2H])([2H])[2H]] 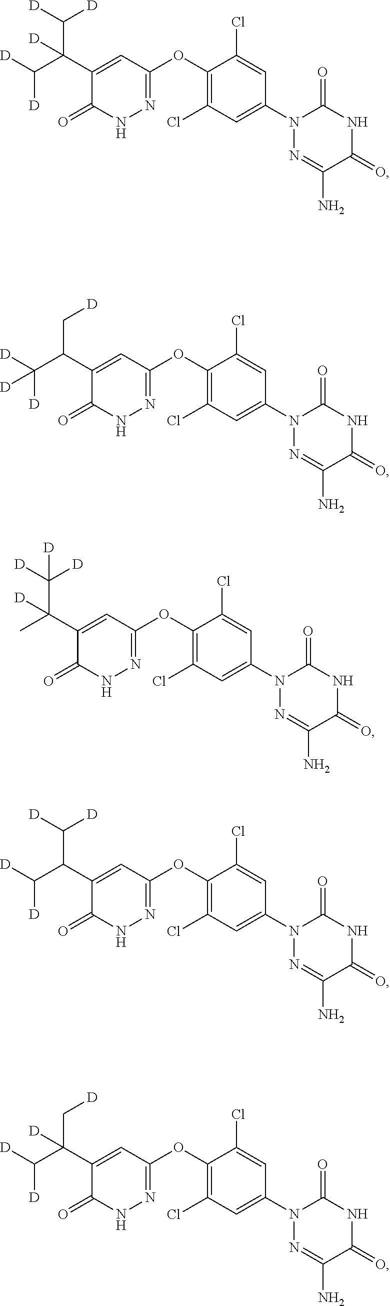 [SMILES: [2H]C([2H])C([2H])(c1cc(Oc2c(Cl)cc(-n3nc(N)c(=O)[nH]c3=O)cc2Cl)n[nH]c1=O)C([2H])[2H] ; [2H]CC(c1cc(Oc2c(Cl)cc(-n3nc(N)c(=O)[nH]c3=O)cc2Cl)n[nH]c1=O)C([2H])([2H])[2H] ; [2H]C([2H])([2H])C([2H])(C)c1cc(Oc2c(Cl)cc(-n3nc(N)c(=O)[nH]c3=O)cc2Cl)n[nH]c1=O ; [2H]C([2H])C(c1cc(Oc2c(Cl)cc(-n3nc(N)c(=O)[nH]c3=O)cc2Cl)n[nH]c1=O)C([2H])[2H] ; [2H]CC([2H])(c1cc(Oc2c(Cl)cc(-n3nc(N)c(=O)[nH]c3=O)cc2Cl)n[nH]c1=O)C([2H])[2H]] 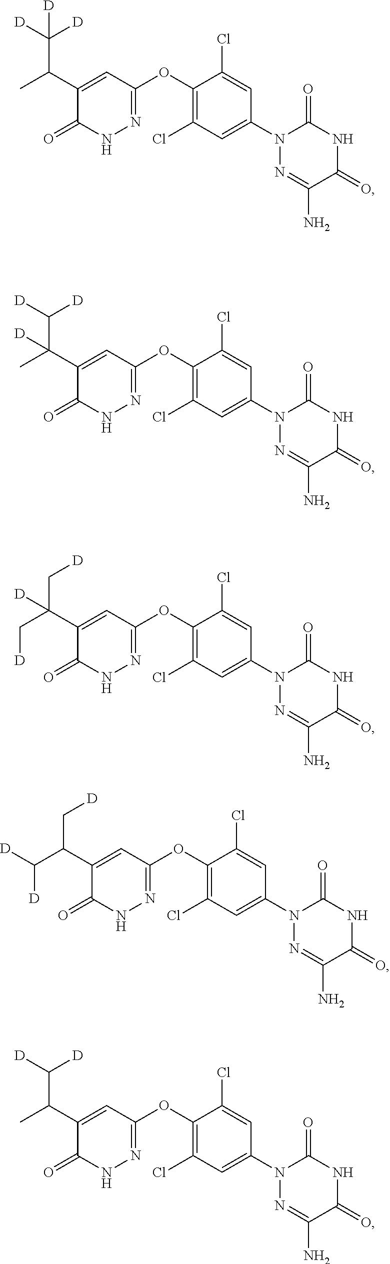 [SMILES: [2H]C([2H])([2H])C(C)c1cc(Oc2c(Cl)cc(-n3nc(N)c(=O)[nH]c3=O)cc2Cl)n[nH]c1=O ; [2H]C([2H])C([2H])(C)c1cc(Oc2c(Cl)cc(-n3nc(N)c(=O)[nH]c3=O)cc2Cl)n[nH]c1=O ; [2H]CC([2H])(C[2H])c1cc(Oc2c(Cl)cc(-n3nc(N)c(=O)[nH]c3=O)cc2Cl)n[nH]c1=O ; [2H]CC(c1cc(Oc2c(Cl)cc(-n3nc(N)c(=O)[nH]c3=O)cc2Cl)n[nH]c1=O)C([2H])[2H] ; [2H]C([2H])C(C)c1cc(Oc2c(Cl)cc(-n3nc(N)c(=O)[nH]c3=O)cc2Cl)n[nH]c1=O] 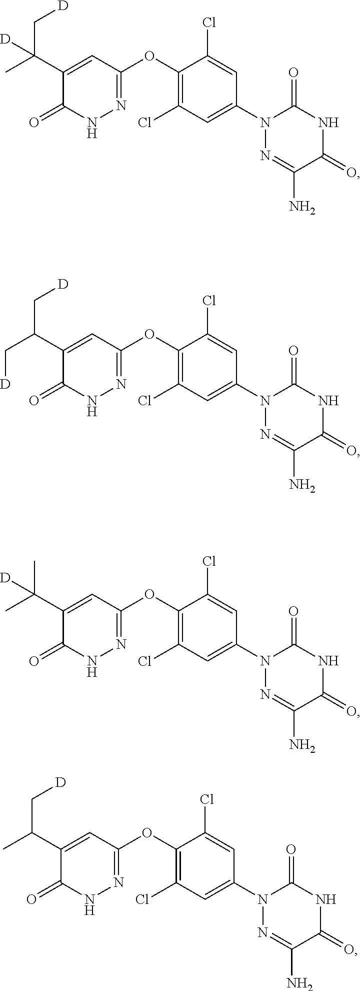 [SMILES: [2H]CC([2H])(C)c1cc(Oc2c(Cl)cc(-n3nc(N)c(=O)[nH]c3=O)cc2Cl)n[nH]c1=O ; [2H]CC(C[2H])c1cc(Oc2c(Cl)cc(-n3nc(N)c(=O)[nH]c3=O)cc2Cl)n[nH]c1=O ; [2H]C(C)(C)c1cc(Oc2c(Cl)cc(-n3nc(N)c(=O)[nH]c3=O)cc2Cl)n[nH]c1=O ; [2H]CC(C)c1cc(Oc2c(Cl)cc(-n3nc(N)c(=O)[nH]c3=O)cc2Cl)n[nH]c1=O] 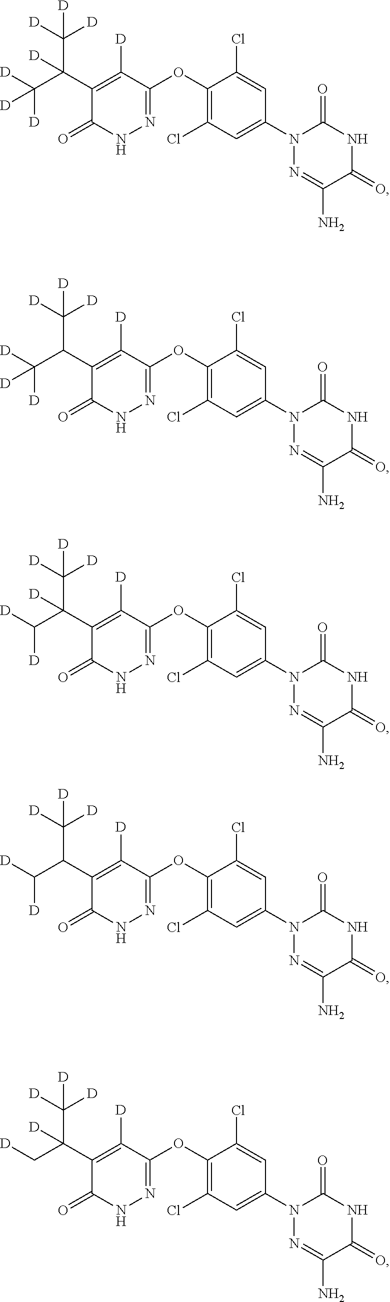 [SMILES: [2H]c1c(Oc2c(Cl)cc(-n3nc(N)c(=O)[nH]c3=O)cc2Cl)n[nH]c(=O)c1C([2H])(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])([2H])[2H])C([2H])([2H])[2H] ; [2H]c1c(Oc2c(Cl)cc(-n3nc(N)c(=O)[nH]c3=O)cc2Cl)n[nH]c(=O)c1C([2H])(C([2H])[2H])C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])([2H])[2H] ; [2H]CC([2H])(c1c([2H])c(Oc2c(Cl)cc(-n3nc(N)c(=O)[nH]c3=O)cc2Cl)n[nH]c1=O)C([2H])([2H])[2H]] 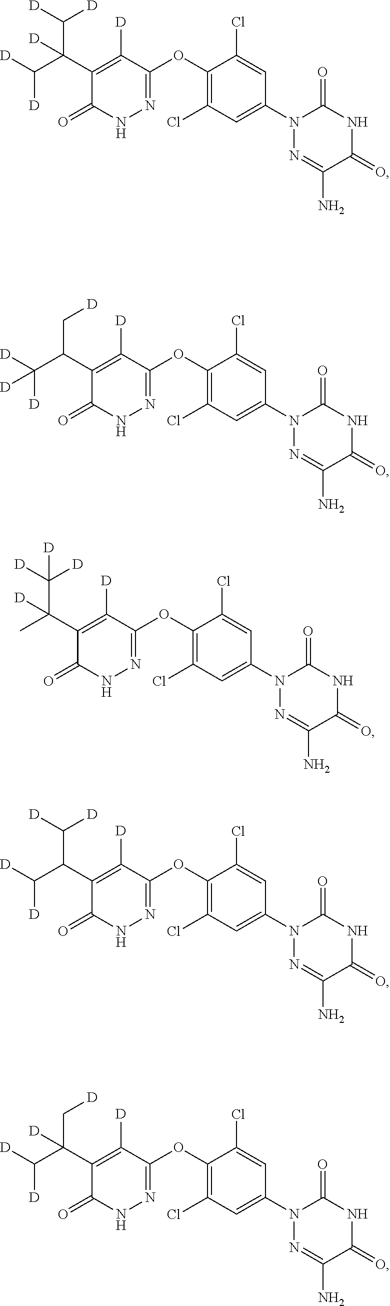 [SMILES: [2H]c1c(Oc2c(Cl)cc(-n3nc(N)c(=O)[nH]c3=O)cc2Cl)n[nH]c(=O)c1C([2H])(C([2H])[2H])C([2H])[2H] ; [2H]CC(c1c([2H])c(Oc2c(Cl)cc(-n3nc(N)c(=O)[nH]c3=O)cc2Cl)n[nH]c1=O)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])([2H])[2H] ; [2H]c1c(Oc2c(Cl)cc(-n3nc(N)c(=O)[nH]c3=O)cc2Cl)n[nH]c(=O)c1C(C([2H])[2H])C([2H])[2H] ; [2H]CC([2H])(c1c([2H])c(Oc2c(Cl)cc(-n3nc(N)c(=O)[nH]c3=O)cc2Cl)n[nH]c1=O)C([2H])[2H]] 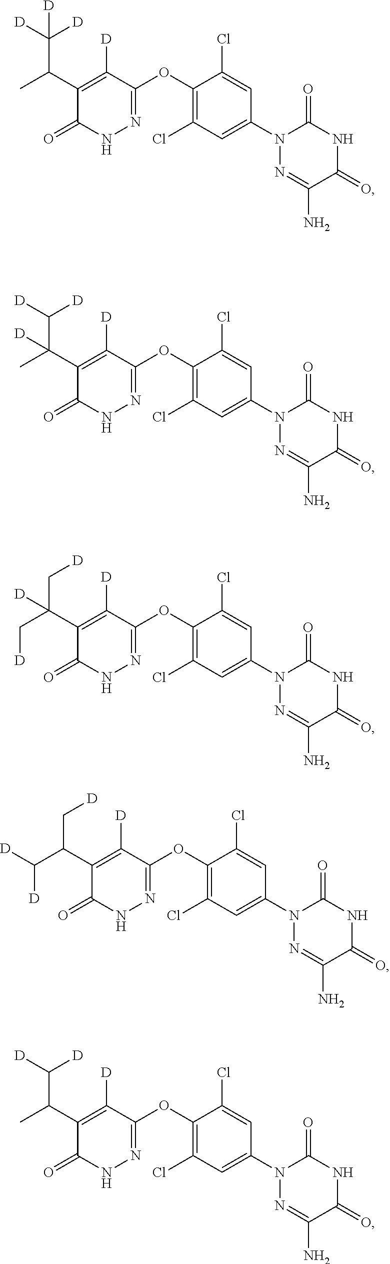 [SMILES: [2H]c1c(Oc2c(Cl)cc(-n3nc(N)c(=O)[nH]c3=O)cc2Cl)n[nH]c(=O)c1C(C)C([2H])([2H])[2H] ; [2H]c1c(Oc2c(Cl)cc(-n3nc(N)c(=O)[nH]c3=O)cc2Cl)n[nH]c(=O)c1C([2H])(C)C([2H])[2H] ; [2H]CC([2H])(C[2H])c1c([2H])c(Oc2c(Cl)cc(-n3nc(N)c(=O)[nH]c3=O)cc2Cl)n[nH]c1=O ; [2H]CC(c1c([2H])c(Oc2c(Cl)cc(-n3nc(N)c(=O)[nH]c3=O)cc2Cl)n[nH]c1=O)C([2H])[2H] ; [2H]c1c(Oc2c(Cl)cc(-n3nc(N)c(=O)[nH]c3=O)cc2Cl)n[nH]c(=O)c1C(C)C([2H])[2H]] 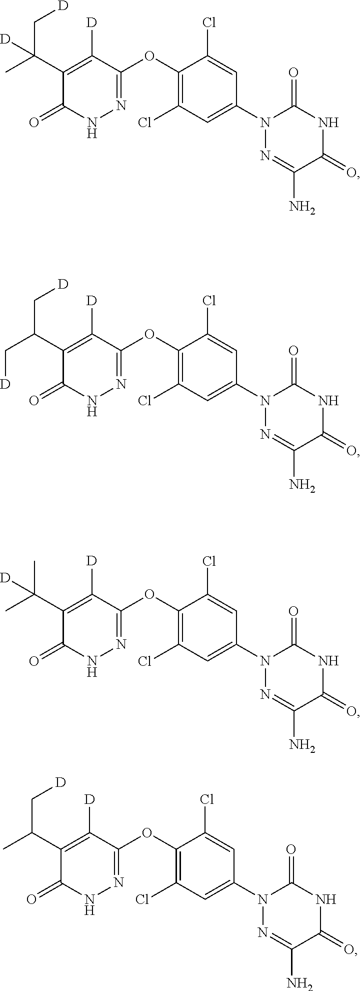 [SMILES: [2H]CC([2H])(C)c1c([2H])c(Oc2c(Cl)cc(-n3nc(N)c(=O)[nH]c3=O)cc2Cl)n[nH]c1=O ; [2H]CC(C[2H])c1c([2H])c(Oc2c(Cl)cc(-n3nc(N)c(=O)[nH]c3=O)cc2Cl)n[nH]c1=O ; [2H]c1c(Oc2c(Cl)cc(-n3nc(N)c(=O)[nH]c3=O)cc2Cl)n[nH]c(=O)c1C([2H])(C)C ; [2H]CC(C)c1c([2H])c(Oc2c(Cl)cc(-n3nc(N)c(=O)[nH]c3=O)cc2Cl)n[nH]c1=O] 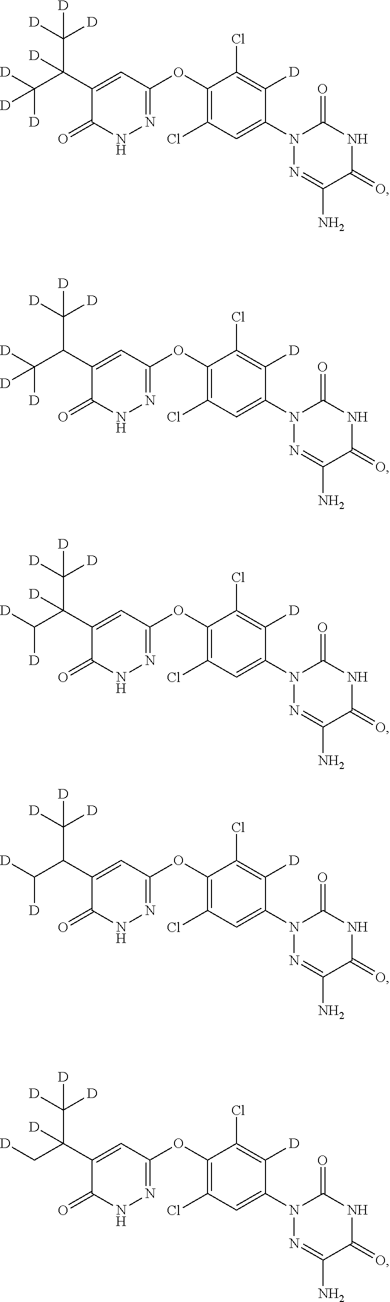 [SMILES: [2H]c1c(-n2nc(N)c(=O)[nH]c2=O)cc(Cl)c(Oc2cc(C([2H])(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])([2H])[2H])C([2H])([2H])[2H])c(=O)[nH]n2)c1Cl ; [2H]c1c(-n2nc(N)c(=O)[nH]c2=O)cc(Cl)c(Oc2cc(C([2H])(C([2H])[2H])C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])([2H])[2H]] 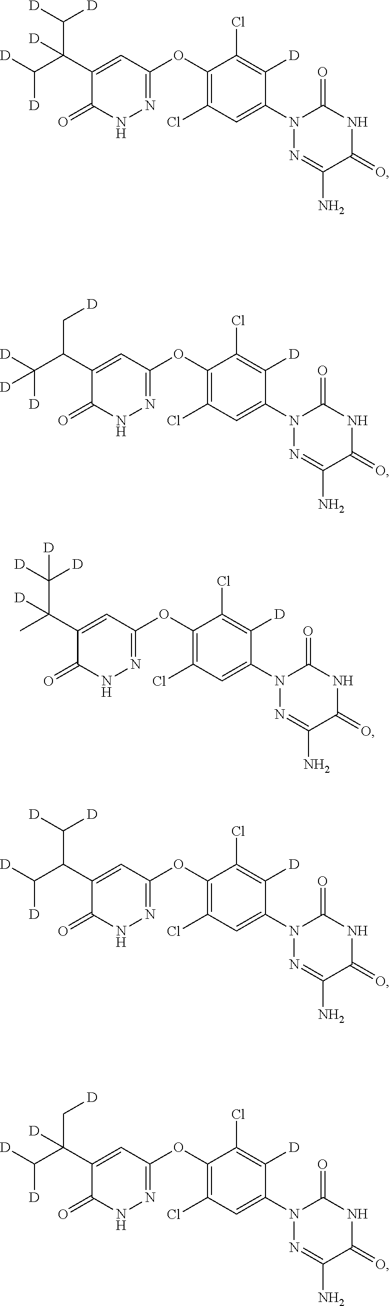 [SMILES: [2H]c1c(-n2nc(N)c(=O)[nH]c2=O)cc(Cl)c(Oc2cc(C([2H])(C([2H])[2H])C([2H])[2H])c(=O)[nH]n2)c1Cl ; [2H]CC(c1cc(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2cc(C([2H])(C)C([2H])([2H])[2H])c(=O)[nH]n2)c1Cl ; [2H]c1c(-n2nc(N)c(=O)[nH]c2=O)cc(Cl)c(Oc2cc(C(C([2H])[2H])C([2H])[2H])c(=O)[nH]n2)c1Cl ; [2H]CC([2H])(c1cc(Oc2c(Cl)cc(-n3nc(N)c(=O)[nH]c3=O)c([2H])c2Cl)n[nH]c1=O)C([2H])[2H]] 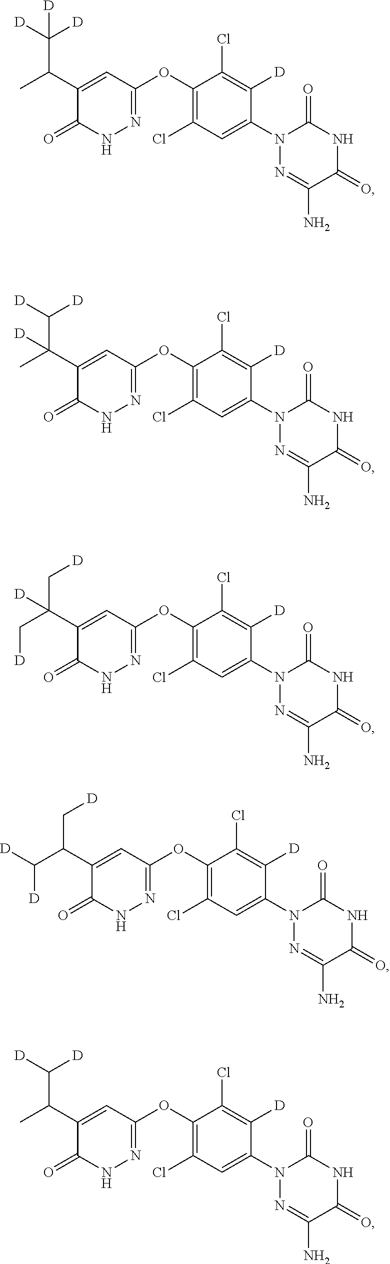 [SMILES: [2H]c1c(-n2nc(N)c(=O)[nH]c2=O)cc(Cl)c(Oc2cc(C(C)C([2H])([2H])[2H])c(=O)[nH]n2)c1Cl ; [2H]c1c(-n2nc(N)c(=O)[nH]c2=O)cc(Cl)c(Oc2cc(C([2H])(C)C([2H])[2H])c(=O)[nH]n2)c1Cl ; [2H]CC([2H])(C[2H])c1cc(Oc2c(Cl)cc(-n3nc(N)c(=O)[nH]c3=O)c([2H])c2Cl)n[nH]c1=O ; [2H]CC(c1cc(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2cc(C(C)C([2H])[2H])c(=O)[nH]n2)c1Cl] 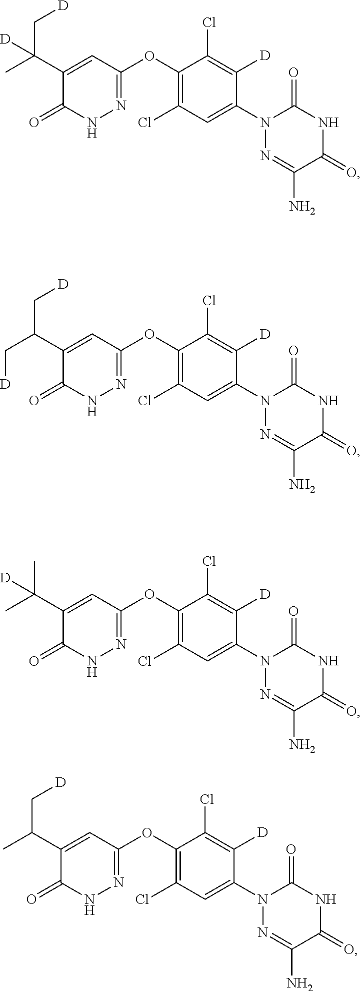 [SMILES: [2H]CC([2H])(C)c1cc(Oc2c(Cl)cc(-n3nc(N)c(=O)[nH]c3=O)c([2H])c2Cl)n[nH]c1=O ; [2H]CC(C[2H])c1cc(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2cc(C([2H])(C)C)c(=O)[nH]n2)c1Cl ; [2H]CC(C)c1cc(Oc2c(Cl)cc(-n3nc(N)c(=O)[nH]c3=O)c([2H])c2Cl)n[nH]c1=O] 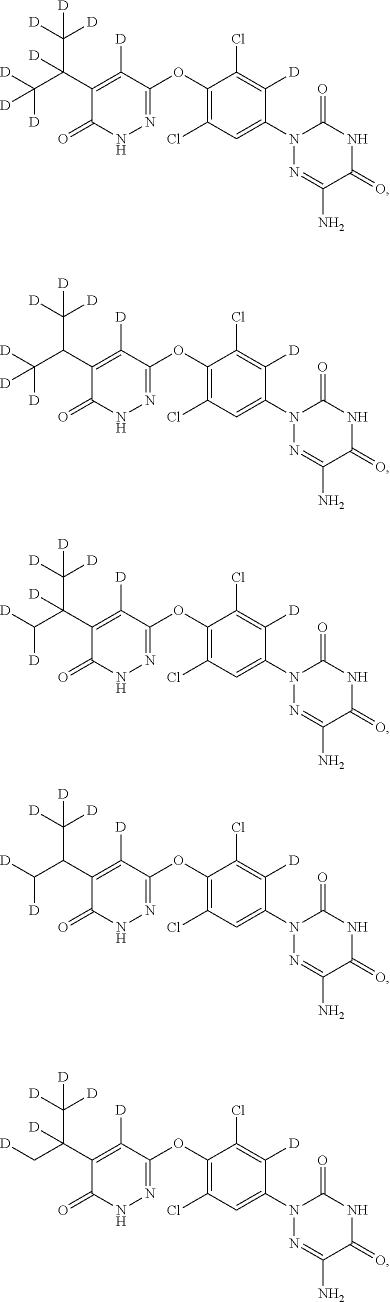 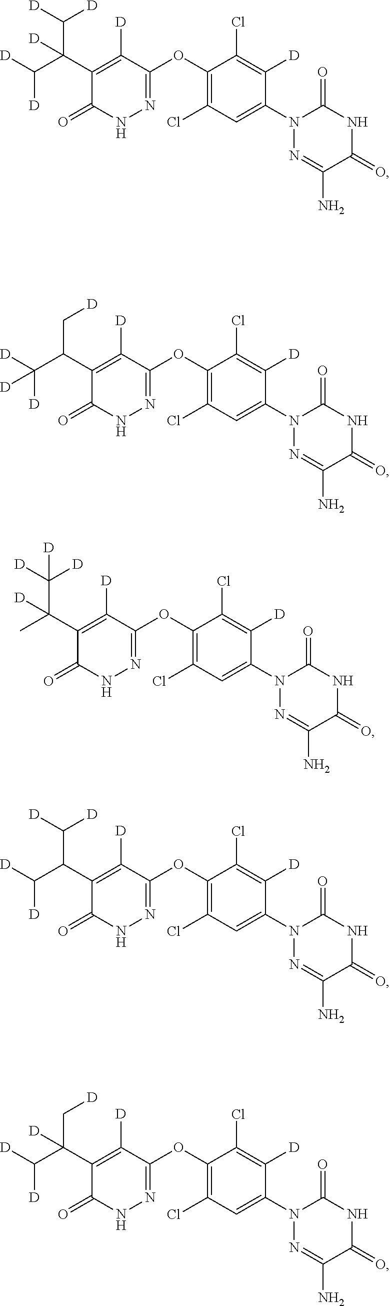 [SMILES: [2H]c1c(-n2nc(N)c(=O)[nH]c2=O)cc(Cl)c(Oc2n[nH]c(=O)c(C([2H])(C([2H])[2H])C([2H])[2H])c2[2H])c1Cl ; [2H]CC(c1c([2H])c(Oc2c(Cl)cc(-n3nc(N)c(=O)[nH]c3=O)c([2H])c2Cl)n[nH]c1=O)C([2H])([2H])[2H] ; [2H]c1c(-n2nc(N)c(=O)[nH]c2=O)cc(Cl)c(Oc2n[nH]c(=O)c(C([2H])(C)C([2H])([2H])[2H])c2[2H])c1Cl ; [2H]c1c(-n2nc(N)c(=O)[nH]c2=O)cc(Cl)c(Oc2n[nH]c(=O)c(C(C([2H])[2H])C([2H])[2H])c2[2H])c1Cl ; [2H]CC([2H])(c1c([2H])c(Oc2c(Cl)cc(-n3nc(N)c(=O)[nH]c3=O)c([2H])c2Cl)n[nH]c1=O)C([2H])[2H]] 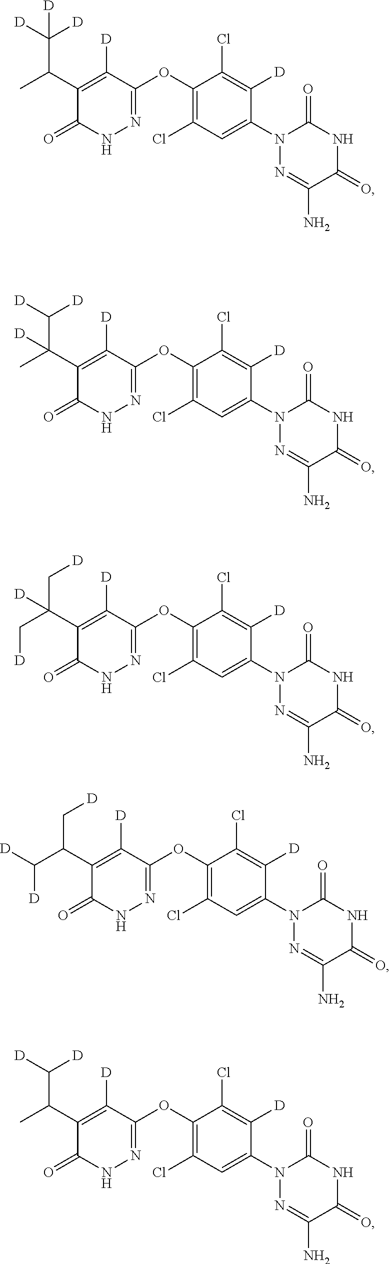 [SMILES: [2H]c1c(-n2nc(N)c(=O)[nH]c2=O)cc(Cl)c(Oc2n[nH]c(=O)c(C(C)C([2H])([2H])[2H])c2[2H])c1Cl ; [2H]c1c(-n2nc(N)c(=O)[nH]c2=O)cc(Cl)c(Oc2n[nH]c(=O)c(C([2H])(C)C([2H])[2H])c2[2H])c1Cl ; [2H]CC([2H])(C[2H])c1c([2H])c(Oc2c(Cl)cc(-n3nc(N)c(=O)[nH]c3=O)c([2H])c2Cl)n[nH]c1=O ; [2H]CC(c1c([2H])c(Oc2c(Cl)cc(-n3nc(N)c(=O)[nH]c3=O)c([2H])c2Cl)n[nH]c1=O)C([2H])[2H] ; [2H]c1c(-n2nc(N)c(=O)[nH]c2=O)cc(Cl)c(Oc2n[nH]c(=O)c(C(C)C([2H])[2H])c2[2H])c1Cl] 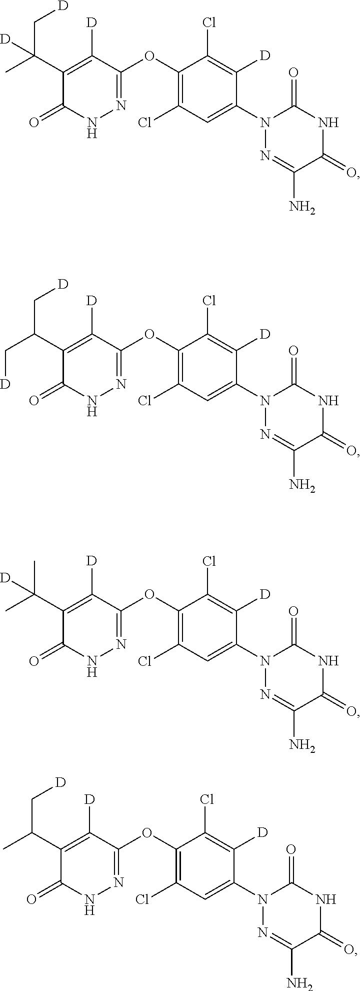 [SMILES: [2H]CC([2H])(C)c1c([2H])c(Oc2c(Cl)cc(-n3nc(N)c(=O)[nH]c3=O)c([2H])c2Cl)n[nH]c1=O ; [2H]CC(C[2H])c1c([2H])c(Oc2c(Cl)cc(-n3nc(N)c(=O)[nH]c3=O)c([2H])c2Cl)n[nH]c1=O ; [2H]c1c(-n2nc(N)c(=O)[nH]c2=O)cc(Cl)c(Oc2n[nH]c(=O)c(C([2H])(C)C)c2[2H])c1Cl ; [2H]CC(C)c1c([2H])c(Oc2c(Cl)cc(-n3nc(N)c(=O)[nH]c3=O)c([2H])c2Cl)n[nH]c1=O] 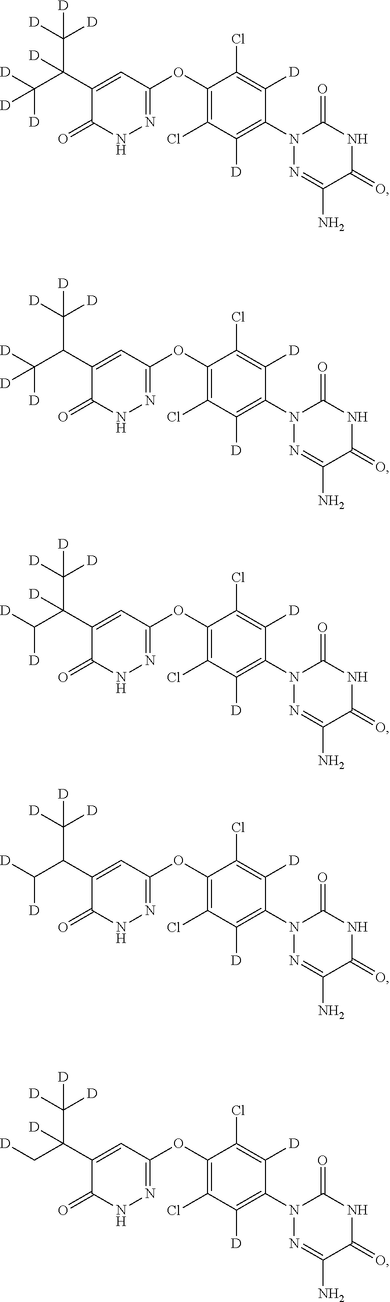 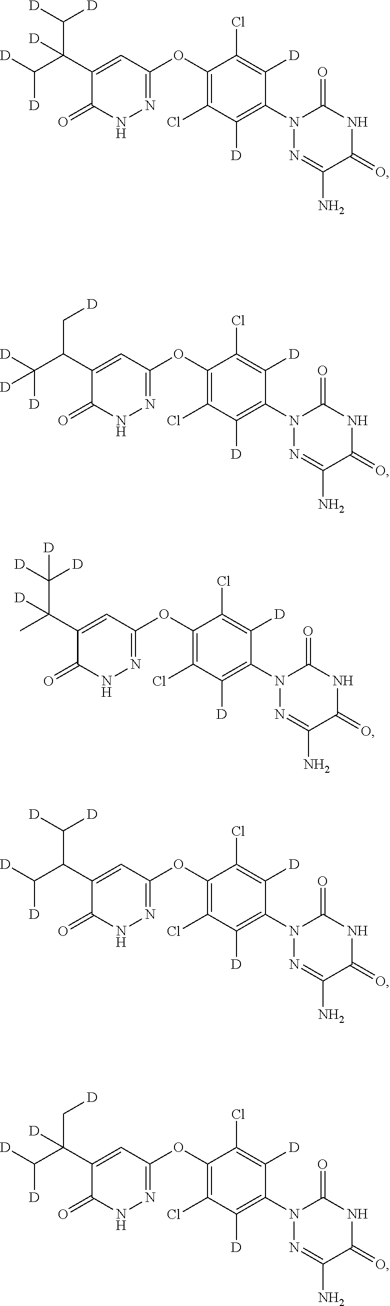 [SMILES: [2H]c1c(Cl)c(Oc2cc(C([2H])(C([2H])[2H])C([2H])[2H])c(=O)[nH]n2)c(Cl)c([2H])c1-n1nc(N)c(=O)[nH]c1=O ; [2H]CC(c1cc(Oc2c(Cl)c([2H])c(-n3nc(N)c(=O)[nH]c3=O)c([2H])c2Cl)n[nH]c1=O)C([2H])([2H])[2H] ; [2H]c1c(Cl)c(Oc2cc(C([2H])(C)C([2H])([2H])[2H])c(=O)[nH]n2)c(Cl)c([2H])c1-n1nc(N)c(=O)[nH]c1=O ; [2H]c1c(Cl)c(Oc2cc(C(C([2H])[2H])C([2H])[2H])c(=O)[nH]n2)c(Cl)c([2H])c1-n1nc(N)c(=O)[nH]c1=O ; [2H]CC([2H])(c1cc(Oc2c(Cl)c([2H])c(-n3nc(N)c(=O)[nH]c3=O)c([2H])c2Cl)n[nH]c1=O)C([2H])[2H]] 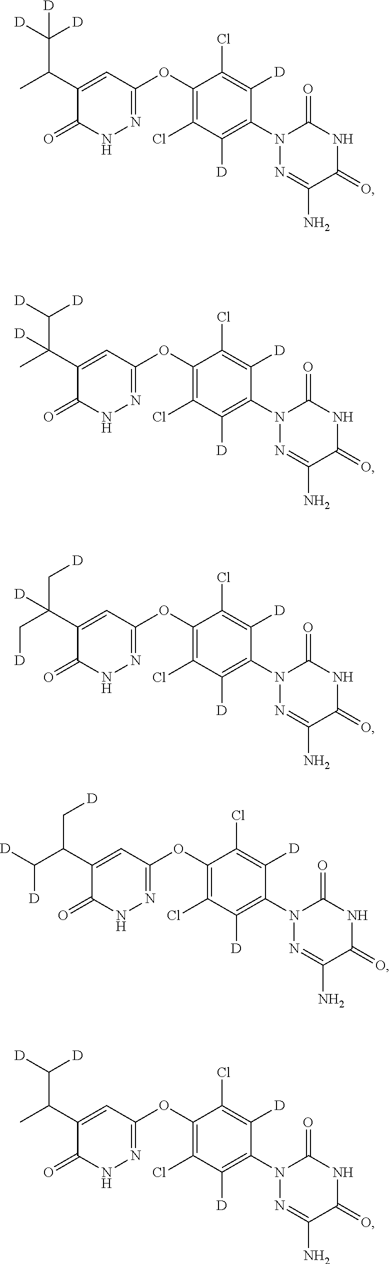 [SMILES: [2H]c1c(Cl)c(Oc2cc(C(C)C([2H])([2H])[2H])c(=O)[nH]n2)c(Cl)c([2H])c1-n1nc(N)c(=O)[nH]c1=O ; [2H]c1c(Cl)c(Oc2cc(C([2H])(C)C([2H])[2H])c(=O)[nH]n2)c(Cl)c([2H])c1-n1nc(N)c(=O)[nH]c1=O ; [2H]CC([2H])(C[2H])c1cc(Oc2c(Cl)c([2H])c(-n3nc(N)c(=O)[nH]c3=O)c([2H])c2Cl)n[nH]c1=O ; [2H]CC(c1cc(Oc2c(Cl)c([2H])c(-n3nc(N)c(=O)[nH]c3=O)c([2H])c2Cl)n[nH]c1=O)C([2H])[2H] ; [2H]c1c(Cl)c(Oc2cc(C(C)C([2H])[2H])c(=O)[nH]n2)c(Cl)c([2H])c1-n1nc(N)c(=O)[nH]c1=O] 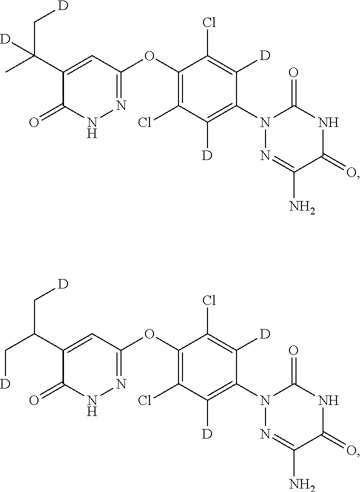 [SMILES: [2H]CC([2H])(C)c1cc(Oc2c(Cl)c([2H])c(-n3nc(N)c(=O)[nH]c3=O)c([2H])c2Cl)n[nH]c1=O ; [2H]CC(C[2H])c1cc(Oc2c(Cl)c([2H])c(-n3nc(N)c(=O)[nH]c3=O)c([2H])c2Cl)n[nH]c1=O] 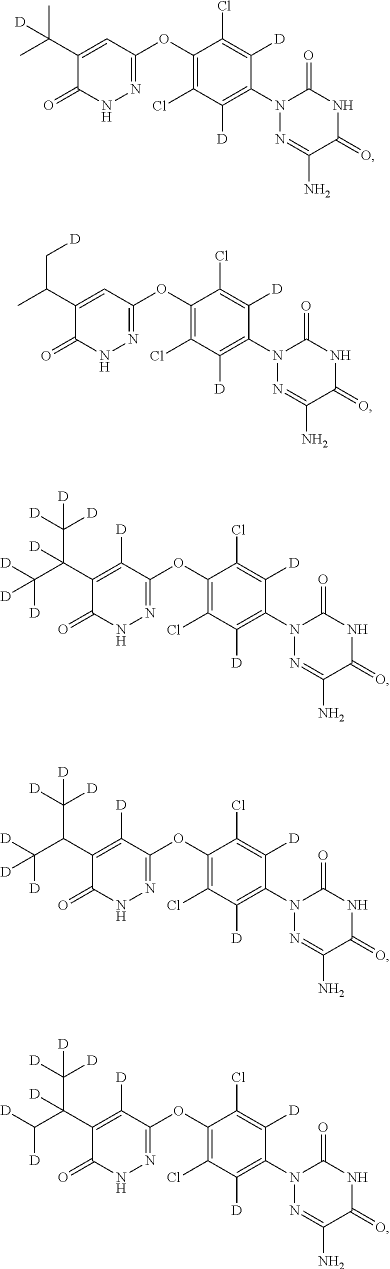 [SMILES: [2H]c1c(Cl)c(Oc2cc(C([2H])(C)C)c(=O)[nH]n2)c(Cl)c([2H])c1-n1nc(N)c(=O)[nH]c1=O ; [2H]CC(C)c1cc(Oc2c(Cl)c([2H])c(-n3nc(N)c(=O)[nH]c3=O)c([2H])c2Cl)n[nH]c1=O ; [2H]c1c(Cl)c(Oc2n[nH]c(=O)c(C([2H])(C([2H])([2H])[2H])C([2H])([2H])[2H])c2[2H])c(Cl)c([2H])c1-n1nc(N)c(=O)[nH]c1=O ; [2H]c1c(Cl)c(Oc2n[nH]c(=O)c(C(C([2H])([2H])[2H])C([2H])([2H])[2H])c2[2H])c(Cl)c([2H])c1-n1nc(N)c(=O)[nH]c1=O ; [2H]c1c(Cl)c(Oc2n[nH]c(=O)c(C([2H])(C([2H])[2H])C([2H])([2H])[2H])c2[2H])c(Cl)c([2H])c1-n1nc(N)c(=O)[nH]c1=O] 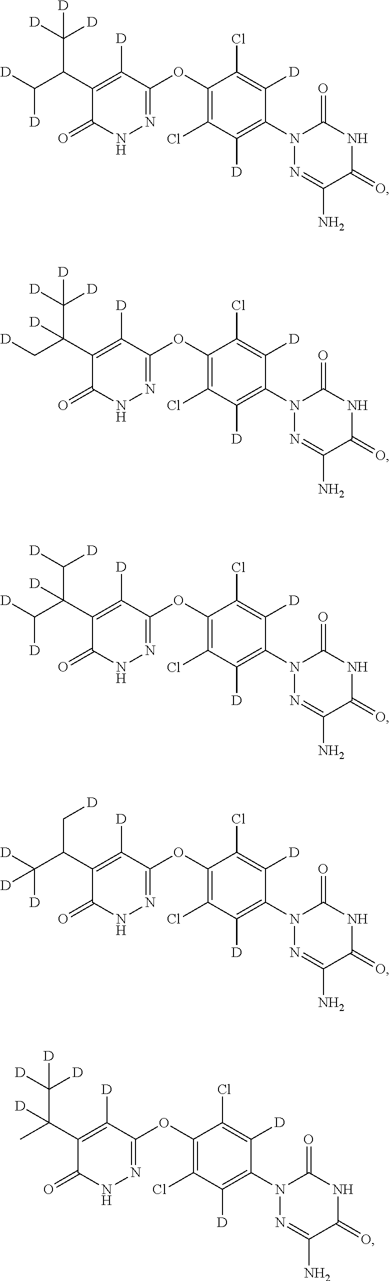 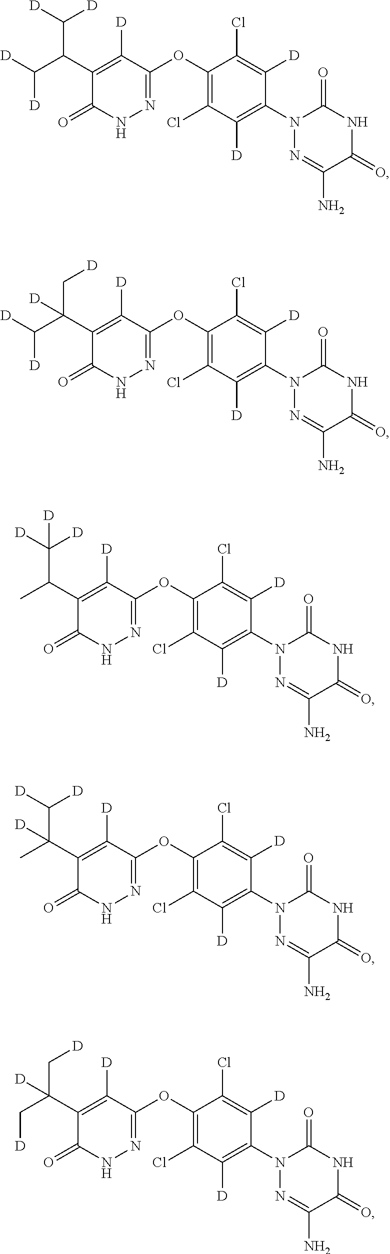 [SMILES: [2H]c1c(Cl)c(Oc2n[nH]c(=O)c(C(C([2H])[2H])C([2H])[2H])c2[2H])c(Cl)c([2H])c1-n1nc(N)c(=O)[nH]c1=O ; [2H]CC([2H])(c1c([2H])c(Oc2c(Cl)c([2H])c(-n3nc(N)c(=O)[nH]c3=O)c([2H])c2Cl)n[nH]c1=O)C([2H])[2H] ; [2H]c1c(Cl)c(Oc2n[nH]c(=O)c(C(C)C([2H])([2H])[2H])c2[2H])c(Cl)c([2H])c1-n1nc(N)c(=O)[nH]c1=O ; [2H]c1c(Cl)c(Oc2n[nH]c(=O)c(C([2H])(C)C([2H])[2H])c2[2H])c(Cl)c([2H])c1-n1nc(N)c(=O)[nH]c1=O ; [2H]CC([2H])(C[2H])c1c([2H])c(Oc2c(Cl)c([2H])c(-n3nc(N)c(=O)[nH]c3=O)c([2H])c2Cl)n[nH]c1=O] 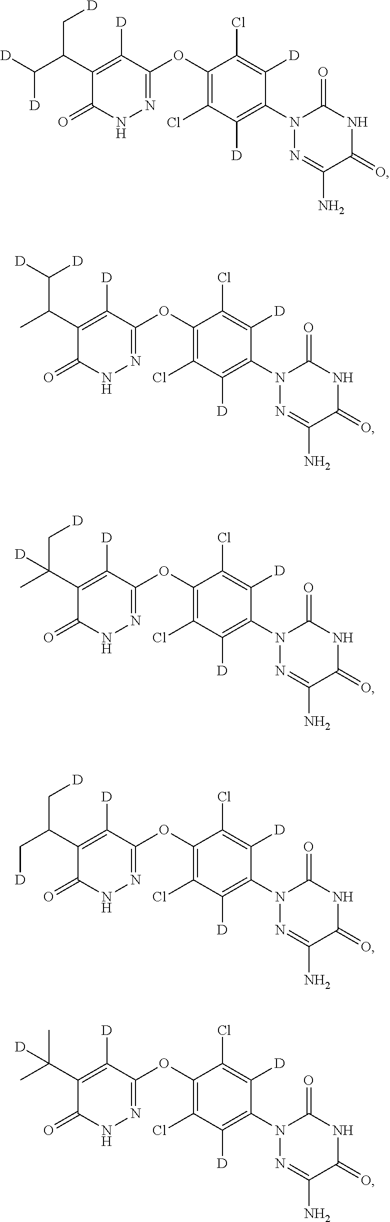 [SMILES: [2H]CC(c1c([2H])c(Oc2c(Cl)c([2H])c(-n3nc(N)c(=O)[nH]c3=O)c([2H])c2Cl)n[nH]c1=O)C([2H])[2H] ; [2H]c1c(Cl)c(Oc2n[nH]c(=O)c(C(C)C([2H])[2H])c2[2H])c(Cl)c([2H])c1-n1nc(N)c(=O)[nH]c1=O ; [2H]CC([2H])(C)c1c([2H])c(Oc2c(Cl)c([2H])c(-n3nc(N)c(=O)[nH]c3=O)c([2H])c2Cl)n[nH]c1=O ; [2H]CC(C[2H])c1c([2H])c(Oc2c(Cl)c([2H])c(-n3nc(N)c(=O)[nH]c3=O)c([2H])c2Cl)n[nH]c1=O ; [2H]c1c(Cl)c(Oc2n[nH]c(=O)c(C([2H])(C)C)c2[2H])c(Cl)c([2H])c1-n1nc(N)c(=O)[nH]c1=O] 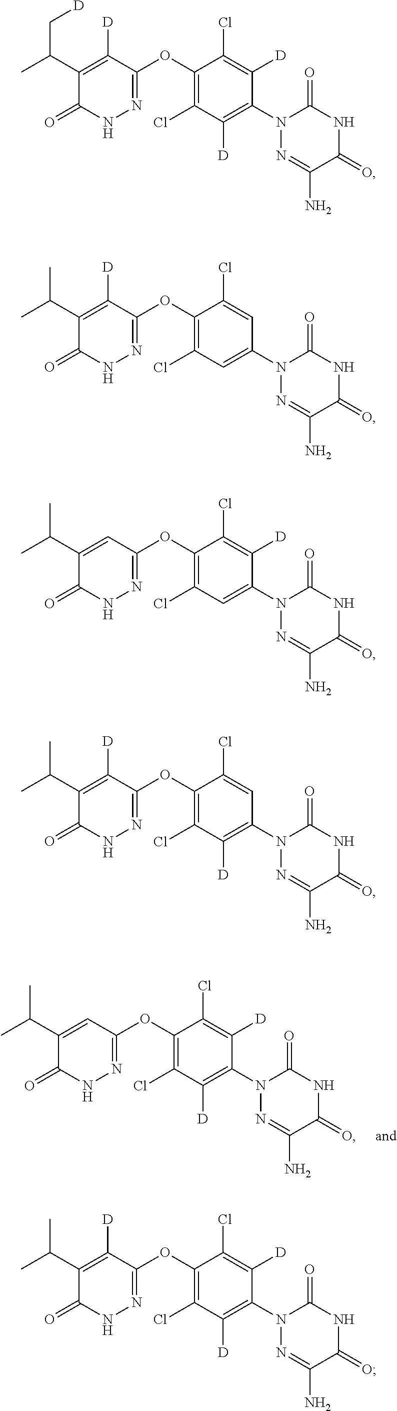 [SMILES: [2H]CC(C)c1c([2H])c(Oc2c(Cl)c([2H])c(-n3nc(N)c(=O)[nH]c3=O)c([2H])c2Cl)n[nH]c1=O ; [2H]c1c(Oc2c(Cl)cc(-n3nc(N)c(=O)[nH]c3=O)cc2Cl)n[nH]c(=O)c1C(C)C ; [2H]c1c(-n2nc(N)c(=O)[nH]c2=O)cc(Cl)c(Oc2cc(C(C)C)c(=O)[nH]n2)c1Cl ; [2H]c1c(-n2nc(N)c(=O)[nH]c2=O)cc(Cl)c(Oc2n[nH]c(=O)c(C(C)C)c2[2H])c1Cl ; [2H]c1c(Cl)c(Oc2cc(C(C)C)c(=O)[nH]n2)c(Cl)c([2H])c1-n1nc(N)c(=O)[nH]c1=O ; [2H]c1c(Cl)c(Oc2n[nH]c(=O)c(C(C)C)c2[2H])c(Cl)c([2H])c1-n1nc(N)c(=O)[nH]c1=O]
or a stereoisomer or a tautomer thereof, or a pharmaceutically acceptable salt of the compound, the stereoisomer, or the tautomer.

6. The compound of claim 5, wherein the compound is 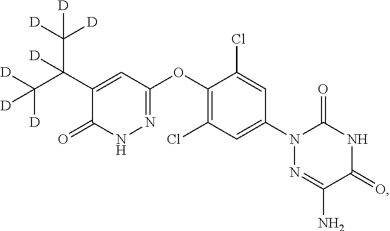 [SMILES: [2H]C([2H])([2H])C([2H])(c1cc(Oc2c(Cl)cc(-n3nc(N)c(=O)[nH]c3=O)cc2Cl)n[nH]c1=O)C([2H])([2H])[2H]]
or the stereoisomer or the tautomer thereof, or the pharmaceutically acceptable salt of the compound, the stereoisomer, or the tautomer.

7. A pharmaceutical composition comprising the compound, stereoisomer, tautomer, or pharmaceutically acceptable salt of claim 1, and at least one pharmaceutically acceptable excipient.

8. A method of treating a disorder or disease in a subject in need thereof, the method comprising administering to the subject a therapeutically effective amount of the compound, stereoisomer, tautomer, or pharmaceutically acceptable salt of claim 1, wherein the disorder or disease is selected from metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

9. The method of claim 8, wherein the compound, stereoisomer, tautomer, or pharmaceutically acceptable salt of claim 1 is administered in combination with a KHK inhibitor, an FXR agonist, a SSAO inhibitor, a FASN inhibitor, or a SCD1 modulator.

10. The method of claim 9, wherein the KHK inhibitor is PF-06835919; the FXR agonist is TERN-101 (LY2562175), tropifexor, obeticholic acid (OCA), or ASC42; the SSAO inhibitor is TERN-201; the FASN inhibitor is ASC40; and the SCD1 modulator is aramchol.

11. (canceled)

12. (canceled)

13. (canceled)

14. A method of selectively modulating the activity of a thyroid hormone receptor beta (THR-β) comprising contacting the compound, stereoisomer, tautomer, or pharmaceutically acceptable salt of claim 1 with the thyroid hormone receptor.

15. The method of claim 14, wherein the contacting is in vitro or ex vivo.

16. The method of claim 14, wherein the contacting is in vivo.

17. (canceled)

18. (canceled)

19. A method of treating a disorder or disease in a subject in need thereof, the method comprising administering to the subject a therapeutically effective amount of the pharmaceutical composition of claim 7, wherein the disorder or disease is selected from metabolic dysfunction dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

20. The method of claim 19, wherein the pharmaceutical composition of claim 7 is administered in combination with a KHK inhibitor, an FXR agonist, a SSAO inhibitor, a FASN inhibitor, or a SCD1 modulator.

21. The method of claim 20, wherein the KHK inhibitor is PF-06835919; the FXR agonist is TERN-101 (LY2562175), tropifexor, obeticholic acid (OCA), or ASC42; the SSAO inhibitor is TERN-201; the FASN inhibitor is ASC40; and the SCD1 modulator is aramchol.
